SEQUENCE LISTING (18 sequences)

SEQ ID NO: 1 - protein, 276 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQYAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 2 - protein, 100 aa, Homo sapiens [sequence omitted]

SEQ ID NO: 3 - protein, 120 aa, Mus musculus
QVQLKQSGPGLVQPSQSLSITCTVSGFSLTSYGLHWVRQSPGKGLEWLGVIWSGGSTDYNAAFISRLSIRKDNSKSQVFFKMNSLQANDTAIYYCARSLTTATSAWFPYWGQGTLVTVSA

SEQ ID NO: 4 - protein, 7 aa, Mus musculus
FSLTSYG

SEQ ID NO: 5 - protein, 5 aa, Mus musculus
WSGGS

SEQ ID NO: 6 - protein, 11 aa, Mus musculus
SLTTATSAWFP

SEQ ID NO: 7 - protein, 120 aa, Mus musculus
SIVMTQTPKFLLVSAGDRVTITCKASQTVSNDVTWYQQKPGQSPKLLIYYASNRYTGVPDRFTGSGYGTDFTFTINTVQAEDLAVYFCQQDYSSPFTFGSGTKLEKKRADAAPTVSIFPP

SEQ ID NO: 8 - protein, 8 aa, Mus musculus
QTVSNDVT

SEQ ID NO: 9 - protein, 7 aa, Mus musculus
YASNRYT

SEQ ID NO: 10 - protein, 9 aa, Mus musculus
QQDYSSPFT

SEQ ID NO: 11 - protein, 120 aa, Mus musculus
QVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSA

SEQ ID NO: 12 - protein, 7 aa, Mus musculus
FSLTSYG

SEQ ID NO: 13 - protein, 5 aa, Mus musculus
WSGGS

SEQ ID NO: 14 - protein, 11 aa, Mus musculus
TFTTSTSAWFA

SEQ ID NO: 15 - protein, 120 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPP

SEQ ID NO: 16 - protein, 8 aa, Mus musculus
ENVVTYVS

SEQ ID NO: 17 - protein, 7 aa, Mus musculus
GASNRYT

SEQ ID NO: 18 - protein, 9 aa, Mus musculus
GQGYSYPYT

(19) United States
(12) Patent Application Publication
(10) Pub. No.: US 2024/0409640 A1
(43) Pub. Date: Dec. 12, 2024

(54) AUGMENTATION OF INNATE AND ADAPTIVE IMMUNITY BY INHIBITION OF INTERACTION OF LILRBS WITH MHC-1

(71) Applicant: The United states of America, as represented by the Secretary, Department of Health, Bethesda, MD (US)

(72) Inventors: Ethan Menahem Shevach, Rockville, MD (US); Abir Kumar Panda, Ellicott City, MD (US); David Harvey Margulies, Bethesda, MD (US); Kannan Natarajan, Rockville, MD (US)

(21) Appl. No.: 18/699,057

(22) PCT Filed: Oct. 5, 2022
(86) PCT No.: PCT/US2022/077634

§ 371 Date: Apr. 5, 2024

Related U.S. Application Data
(60) Provisional application No. 63/262,120, filed on Oct. 5, 2021.

(51) Int. Cl. C07K 16/28; A61K 39/00; A61P 37/04

(52) U.S. Cl. CPC C07K 16/2833; A61P 37/04; A61K 2039/505; C07K 2317/565; C07K 2317/74; C07K 2317/76

(57) ABSTRACT

Methods for regulating an immune response are described. More specifically, methods are described in which a compound is used to inhibit the interaction of an MHC class I molecule with a leukocyte immunoglobulin-like receptor B (LILRB) protein. Such inhibition results in activation of immune cells expressing the LILRB protein. Also described are uses of such methods for treating disease.

This application relates to and claims priority to U.S. Provisional Patent Application No. 63/262,120, which was filed on Oct. 5, 2021, and is incorporated herein by reference in its entirety.

### STATEMENT REGARDING FEDERALLY SPONSORED RESEARCH & DEVELOPMENT

This invention was made with government support under the Intramural Program of the National Institute of Allergy and Infectious Diseases Project Number Z01-A11000959. The government has certain rights in the invention.

### FIELD OF THE INVENTION

The disclosure relates to methods of modulating the immune system in order to treat or prevent disease. More specifically, the disclosure relates to methods of preventing and/or treating disease by inhibiting the interaction of cells expressing leukocyte immunoglobulin-like receptors (LILRs) with human Major Histocompatibility Complex (MHC) class I human leukocyte antigen (HLA) molecules.

### BACKGROUND OF THE INVENTION

The immune system has evolved to protect individuals from infection by microorganisms such as bacteria, viruses, and parasites. The immune system has also evolved so that is able to identify cells as either self or non-self, an ability that plays an important role in tumor surveillance. Over the last 50 years, numerous cells and pathways have been identified that are involved in these functions of the immune system. Generally, these cells and pathways are divided into the innate immune system, and the adaptive immune system.

Innate immunity is generally viewed as “the first line of defense” against pathogens and represents a crucial part of the systemic response to prevent infection. In this regard, the innate immune system has the capacity to produce an induced response during a primary infection, and to create inflammatory conditions to contain the infection. The innate immune system also plays a critical role in the activation and regulation of adaptive immunity.

Elements of the innate immune system include external physical barriers, and humoral and cellular effector mechanisms that aid in identifying foreign molecules, and also function to determine self from non-self. These cellular effector mechanisms include protein complexes, such as major histocompatibility complex I (MHC)-I that is expressed on the surface of cells. These MHC-I complexes present intracellular peptides to cells of the immune system, which is a key method for determining self from non-self. In addition, co-stimulatory and co-inhibitory signals between the MHC-I complex and cells of the immune system are communicated through proteins expressed on immune cells. Such interactions are referred to as checkpoints, since they “check” whether the immune system should be active or suppressed. Examples of such signaling proteins include natural cytotoxicity receptors (NCRs), Killer-cell immunoglobulin-like receptors (KIRs), and leukocyte immunoglobulin-like receptors (LILRs).

Based on the biological properties of such molecules, various therapeutics strategies have been developed to modulate these checkpoints, and the resulting therapeutic compounds have been termed “checkpoint inhibitors”. While some promising results have been obtained, many checkpoints inhibitors are too specific, affecting only limited pathways in the immune system. Moreover, compounds are still not available for many components of checkpoints. Thus, there is still a need for compounds that target novel checkpoint components, and that have broader activity. The present application addresses this need and provides new options for modulating the immune system.

### SUMMARY

The present disclosure provides a method for augmenting innate and adaptive immunity by inhibiting the interaction of a leukocyte immunoglobulin-like receptor B (LILRB) protein with an MHC class I human leukocyte antigen (HLA) molecule. The method may be performed in vitro or in vivo. One aspect provides a method comprising administering to a human subject a compound that recognizes a binding site on an MHC Class I HLA molecule, wherein binding of the compound to the binding site on the MHC Class I HLA molecule inhibits interaction of the MHC Class I HLA molecule with a LILRB protein; and, wherein binding of the compound to the binding site on the MHC Class I HLA molecule does not affect interaction of the MHC Class I HLA molecule and a T-cell receptor (TCR).

One aspect provides a method of preventing a LILRB protein from binding to a MHC class I HLA molecule, comprising contacting the MHC class I HLA molecule with a compound that recognizes a binding site on the MHC class I HLA molecule; wherein binding of the compound to the binding site on the MHC class I HLA molecule inhibits interaction of the MHC class I HLA molecule with the LILRB protein; and, wherein binding of the compound to the binding site on the MHC class I HLA molecule does not affect the interaction between the MHC class I HLA molecule and a TCR. The LILRB and the MHC class I HLA molecule may in a subject. The subject may be a human subject. The compound may be administered to the subject using a route of administration that allows the compound to contact the MHC class I HLA molecule, which may include oral or parenteral administration, which may be achieved by injection. The method may be performed in vitro, where the LILRB protein and the MHC class I HLA molecule may be free-floating or they may be immobilized.

One aspect provides a method of activating a human cell, such as an immune cell, that expresses a LILRB protein, comprising blocking the interaction of the LILRB protein with an MHC class I HLA molecule by contacting the MHC class I HLA molecule with a compound that recognizes a binding site on the HLA molecule; wherein binding of the compound to the binding site on the MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a TCR. The immune cell may be selected from the group consisting of a natural killer (NK) cell, a T-lymphocyte, a B-lymphocyte, a monocyte, a macrophage, a dendritic cell, and a granulocyte. The immune cell and the MHC class I HLA molecule may be in a subject, such as a human subject, or they may be in a vessel, where the LILRB protein and the MHC class I HLA molecule may be free floating or they may be immobilized.

One aspect provides a method of treating cancer in an individual, comprising administering to the individual a compound that recognizes a binding site on an MHC class I HLA molecule; wherein binding of the compound to the binding site on the MHC class I HLA molecule inhibits interaction of the MHC class I HLA molecule with a LILRB protein; and, wherein binding of the compound to the binding site on the MHC class I HLA molecule does not affect the interaction between the MHC class I HLA and a TCR. The cancer may be selected from the group consisting of a sarcoma, a carcinoma, a melanoma, leukemia, lymphoma, a cancers of the breast, head and neck cancer, ovarian cancer, bladder cancer, lung cancer, pharyngeal cancer, laryngeal cancer, esophageal cancer, stomach cancer, intestinal cancer, liver cancer, pancreatic cancer, colon cancer, a cancer of the female reproductive tract, a cancer of the male reproductive tract, prostate cancer, kidney cancer, and a cancer of the central nervous system. The method may comprise administering to the individual at least one additional anti-cancer treatment, which may be check-point inhibitor selected from the group consisting of anti-PD-1, anti-PD-L1, anti-CTLA-4, anti-LAG-3, and anti-TIM3. The at least one additional cancer treatment may be a cytokine, which may be selected from the group consisting of a cytokine that targets the IL-2/IL-2 receptor pathway, a cytokine that targets the IFNAR1/2 pathway, and granulocyte-macrophage colony-stimulating factor (GM-CSF).

One aspect provides a method of treating an infectious disease in an individual, comprising administering to the individual a compound that recognizes a binding site on an MHC class I HLA molecule; wherein binding of the compound to the binding site on the MHC class I HLA inhibits interaction of the MHC class I HLA with a LILRB protein; and, wherein binding of the compound to the binding site on the MHC class I HLA does not affect interaction between the class I HLA molecule and TCR. The infectious disease may be caused by a bacterium, a virus, or a parasite, which may be Mycobacteria tuberculosis, Salmonella typhi, Salmonella typhimurium, Salmonella enteritidis, Listeria monocytogenes, Rickettsia rickettsia, Legionella pneumophila, Human Immunodeficiency Virus (HIV), herpesvirus, hepatitis virus, Varicella-Zoster virus, Epstein Barr virus, respiratory syncytial virus (RSV), papillomavirus, and coronaviruses (including Middle Eastern Respiratory Syndrome virus (MERS), Severe Acute Respiratory Syndrome virus (SARS), or Severe Acute respiratory Syndrome coronavirus 2 (SARS-CoV-2). The method may comprise administering at least one additional anti-bacterial or anti-viral agent.

One aspect provides a method of converting a Th2-mediated immune response in an individual to a Th1-mediated immune response, comprising administering to the individual a compound that recognizes a binding site on an MHC class I HLA molecule; wherein binding of the compound to the binding site on the MHC class I HLA molecule inhibits interaction of the MHC class I HLA molecule with a LILRB protein; and, wherein binding of the compound to the binding site on the MHC class I HLA molecule does not affect the interaction between the MHC class I HLA molecule and a TCR.

One aspect provides a method of treating an individual for a Th2-mediated disease, comprising administering to the individual a compound that recognizes a binding site on an MHC class I HLA molecule; wherein binding of the compound to the binding site on the MHC class I HLA molecule inhibits interaction of the MHC class I HLA molecule with a LILRB protein; and, wherein binding of the compound to the binding site on the MHC class I HLA molecule does not affect the interaction between the MHC class I HLA molecule and a TCR. The Th2-mediated disease may comprise asthma, allergic rhinitis, atopic dermatitis, systemic progressive sclerosis, or Omenn's syndrome.

One aspect provides a method of treating an individual for a Th2-mediated reaction to an allergen, comprising administering to the individual a compound that recognizes a binding site on an MHC class I HLA molecule; wherein binding of the compound to the binding site on the MHC class I HLA molecule inhibits interaction of the MHC class I HLA molecule with a LILRB protein; and, wherein binding of the compound to the binding site on the MHC class I HLA molecule does not affect the interaction between the MHC class I HLA molecule and a TCR. The compound may be administered to the individual at about the same time the individual contacts the allergen, or at a time following contact with the allergen. Contact of the allergen with the individual may be accidental or the allergen may be administered to the individual.

One aspect provides a method of preventing a Th2-mediated reaction to an allergen in an individual, comprising administering to the individual a compound that recognizes a binding site on an MHC class I HLA molecule; wherein binding of the compound to the binding site on the MHC class I HLA molecule inhibits interaction of the MHC class I HLA molecule with a LILRB protein; wherein binding of the compound to the binding site on the MHC class I HLA molecule does not affect the interaction between the MHC class I HLA molecule and a TCR; and, wherein the compound is administered to the individual prior to the individual contacting the allergen.

In these methods, the allergen may be an animal protein, dust, a pharmaceutical agent, a food molecule, an egg protein, a nut protein, a milk protein, a soy protein, a fish protein, a wheat protein, gluten, a strawberry protein, a corn protein, a legume, a milk protein, a fungal spore, insect feces, mite feces, cockroach calyx, insect venom, natural rubber, pollen, or a metal.

One aspect provides a method of augmenting the immunogenicity of a vaccine, comprising co-administering the vaccine and a compound that recognizes a binding site on an MHC class I HLA molecule; wherein binding of the compound to the binding site on the MHC class I HLA molecule inhibits interaction of the MHC class I HLA molecule with a LILRB protein; and, wherein binding of the compound to the binding site on the MHC class I HLA molecule does not affect interaction between the MHC class I HLA molecule and a TCR. In certain aspects, co-administering may comprise administering the compound immediately before, at the same time as, or immediately after administering the vaccine.

In these methods, the MHC class I HLA molecule may be HLA-A, HLA-B, HLA-C, HLA-E, HLA-F, or HLA-G.

In these methods, the compound may bind to a location on the HLA molecule that differs from where the LILRB protein interacts with the MHC class I HLA molecule. The compound may bind to a location on the HLA molecule that overlaps where the LILRB protein binds the MHC class I HLA molecule. The binding site on the HLA molecule may comprise one or more amino acid residues corresponding to one or more amino acid residues selected from the group consisting of Q87 of SEQ ID NO:1, K121 of SEQ ID NO:1, D122 of SEQ ID NO:1, A136 of SEQ ID NO:1, D137 of SEQ ID NO:1, T225 of SEQ ID NO:1, Q226 of SEQ ID NO:1, D227 of SEQ ID NO:1, T228 of SEQ ID NO:1, E232 of SEQ ID NO:1, 11 of SEQ ID NO:2, Q2 of SEQ ID NO:2, R3 of SEQ ID NO:2, K6 of SEQ ID NO:2, K58 of SEQ ID NO:2, D59 of SEQ ID NO:2, and W60 of SEQ ID NO:2. The binding site on the HLA molecule may comprise one or more amino acid residues selected from the group consisting of: a glutamine at a position corresponding to amino acid position 87 of SEQ ID NO:1; a lysine at a position corresponding 122 of SEQ ID NO:1; an aspartic acid at a position corresponding to amino acid position 122 of SEQ ID NO:1; an alanine at a position corresponding to amino acid position 136 of SEQ ID NO:1; an aspartic acid at a position corresponding to amino acid position 137 of SEQ ID NO:1; a threonine at a position corresponding to amino acid position 225 of SEQ ID NO:1; a glutamine at a position corresponding to amino acid position 226 of SEQ ID NO:1; an aspartic acid at a position corresponding to amino acid position 227 of SEQ ID NO:1; a threonine at a position corresponding to amino acid position 228 of SEQ ID NO:1; a glutamic acid at a position corresponding to amino acid position 232 of SEQ ID NO:1; an isoleucine at a position corresponding to amino acid position 1 of SEQ ID NO:2; a glutamine at a position corresponding to amino acid position 2 of SEQ ID NO:2; an arginine at a position corresponding to amino acid position 3 of SEQ ID NO:2; a lysine at a position corresponding to amino acid position 6 of SEQ ID NO:2; a lysine at a position corresponding to amino acid position 58 of SEQ ID NO:2; an aspartic acid at a position corresponding to amino acid position 59 of SEQ ID NO:2; and, a tryptophan at a position corresponding to amino acid position 60 of SEQ ID NO:2. The binding site on the HLA molecule comprise at least 2, at least 3, at least 4, at least 5, at least 6, at least 7, at least 8, at least 9, at least 10, at least 11, at least 12, at least 13, at least 14, at least 15, at least 16, at least 17, or at least 18, amino acid residues selected from the group consisting of: a glutamine at a position corresponding to amino acid position 87 of SEQ ID NO:1; a lysine at a position corresponding 122 of SEQ ID NO:1; an aspartic acid at a position corresponding to amino acid position 122 of SEQ ID NO:1; an alanine at a position corresponding to amino acid position 136 of SEQ ID NO:1; an aspartic acid at a position corresponding to amino acid position 137 of SEQ ID NO:1; a threonine at a position corresponding to amino acid position 225 of SEQ ID NO:1; a glutamine at a position corresponding to amino acid position 226 of SEQ ID NO:1; an aspartic acid at a position corresponding to amino acid position 227 of SEQ ID NO:1; a threonine at a position corresponding to amino acid position 228 of SEQ ID NO:1; a glutamic acid at a position corresponding to amino acid position 232 of SEQ ID NO:1; an isoleucine at a position corresponding to amino acid position 1 of SEQ ID NO:2; a glutamine at a position corresponding to amino acid position 2 of SEQ ID NO:2; an arginine at a position corresponding to amino acid position 3 of SEQ ID NO:2; a lysine at a position corresponding to amino acid position 6 of SEQ ID NO:2; a lysine at a position corresponding to amino acid position 58 of SEQ ID NO:2; an aspartic acid at a position corresponding to amino acid position 59 of SEQ ID NO:2; and, a tryptophan at a position corresponding to amino acid position 60 of SEQ ID NO:2.

The binding site on the HLA molecule may comprise: a glutamine at a position corresponding to amino acid position 87 of SEQ ID NO:1; a lysine at a position corresponding 122 of SEQ ID NO:1; an aspartic acid at a position corresponding to amino acid position 122 of SEQ ID NO:1; an alanine at a position corresponding to amino acid position 136 of SEQ ID NO:1; an aspartic acid at a position corresponding to amino acid position 137 of SEQ ID NO:1; a threonine at a position corresponding to amino acid position 225 of SEQ ID NO:1; a glutamine at a position corresponding to amino acid position 226 of SEQ ID NO:1; an aspartic acid at a position corresponding to amino acid position 227 of SEQ ID NO:1 a threonine at a position corresponding to amino acid position 228 of SEQ ID NO:1; a glutamic acid at a position corresponding to amino acid position 232 of SEQ ID NO:1; an isoleucine at a position corresponding to amino acid position 1 of SEQ ID NO:2; a glutamine at a position corresponding to amino acid position 2 of SEQ ID NO:2; an arginine at a position corresponding to amino acid position 3 of SEQ ID NO:2; a lysine at a position corresponding to amino acid position 6 of SEQ ID NO:2; a lysine at a position corresponding to amino acid position 58 of SEQ ID NO:2; an aspartic acid at a position corresponding to amino acid position 59 of SEQ ID NO:2; and, a tryptophan at a position corresponding to amino acid position 60 of SEQ ID NO:2.

The compound may be a peptide, a protein, an antibody, a small molecule, a DNA molecule, an RNA molecule, or a combination thereof. The antibody may be a human or a humanized antibody, a monoclonal antibody (mAb), or an antigen-binding portion thereof. In certain aspects, binding of the compound to the MHC class I HLA molecule may inhibit binding of monoclonal antibody W6/32 or monoclonal antibody DX17 to the MHC class I HLA molecule. The location of the binding site for the compound may differ from, or overlap with, the location of the binding site for monoclonal antibody W6/32 or monoclonal antibody DX17. The binding site for the compound on the MHC class I HLA molecule may share one or more amino acid residues with the binding site of the monoclonal antibody W6/32 or the monoclonal antibody DX17. In certain aspects, the compound may be an antibody. The antibody may inhibit binding of monoclonal antibody W6/32 or monoclonal antibody DX17 to the MHC class I HLA molecule. The location of the binding site for the antibody may differ from, or overlap with, the location of the binding site for W6/32 monoclonal antibody or DX17 monoclonal antibody. The binding site for the antibody on the MHC class I HLA molecule may share one or more amino acid residues with the binding site of the W6/32 monoclonal antibody or the DX17 monoclonal antibody.

In aspects of the disclosure in which the compound is an antibody, preferably the antibody is not capable of being bound by, and is not capable of binding, an Fc receptor. Thus, the antibody may comprise one or more mutations that result in the antibody being incapable of being bound by, and incapable of binding, an Fc receptor. Examples of such mutations are disclosed herein.

The compound may be an antibody, or an antigen-binding portion thereof, that comprises a heavy chain variable region (VH) comprising VH complementarity determining regions (CDRs) 1, 2, and 3, wherein the CDR1 region has the amino acid sequence of SEQ ID NO:4 or SEQ ID NO:12; wherein the CDR2 region has the amino acid sequence of SEQ ID NO:5 or SEQ ID NO:13; and wherein the CDR3 region has the amino acid sequence of SEQ ID NO:6 or SEQ ID NO:14. The compound may be an antibody, or an antigen-binding portion thereof, that comprises a heavy chain variable region (VH) comprising VH complementarity determining regions (CDRs) 1, 2, and 3, having an amino acid sequences as set forth in SEQ ID NO:4, SEQ ID NO:5, and SEQ ID NO:6, respectively, or as set forth in SEQ ID NO:12, SEQ ID NO:13, and SEQ ID NO:14, respectively. The compound may be an antibody, or an antigen-binding portion thereof, that comprises a light chain variable region (VL) comprising VL complementarity determining regions (CDRs) 1, 2, and 3, wherein the CDR1 region has the amino acid sequence of SEQ ID NO:8 or SEQ ID NO:16; wherein the CDR2 region has the amino acid sequence of SEQ ID NO:9 or SEQ ID NO:17; and wherein the CDR3 region has the amino acid sequence of SEQ ID NO:10 or SEQ ID NO:18. The compound may be an antibody, or an antigen-binding portion thereof, that comprises a light chain variable region (VL) comprising VL complementarity determining regions (CDRs) 1, 2, and 3, having the amino acid sequences set forth in SEQ ID NO:8, SEQ ID NO:9, and SEQ ID NO:10, respectively, or in SEQ ID NO:16, SEQ ID NO:17, and SEQ ID NO:18, respectively. The compound may be an antibody, or an antigen-binding portion thereof, that comprises: a heavy chain variable region (VH) comprising VH complementarity determining regions (CDRs) 1, 2, and 3; and, a light chain variable region (VL) comprising VL complementarity determining regions (CDRs) 1, 2, and 3; wherein the VH CDR1 region has an amino acid sequence selected from SEQ ID NO:4 or SEQ ID NO:12; wherein the VH CDR2 region has an amino acid sequence selected from SEQ ID NO:5 and SEQ ID NO:13; wherein the VH CDR3 region has an amino acid sequence selected from SEQ ID NO:6 and SEQ ID NO:14; wherein the VL CDR1 region has an amino acid sequence selected from SEQ ID NO:8 or SEQ ID NO:16; wherein the VL CDR2 region has an amino acid sequence selected from SEQ ID NO:9 and SEQ ID NO:17; and, wherein the VL CDR3 region has an amino acid sequence selected from SEQ ID NO:10 and SEQ ID NO:18.

The compound may be an antibody, or an antigen-binding portion thereof, that comprises: a heavy chain variable region (VH) comprising VH complementarity determining regions (CDRs) 1, 2, and 3; and, a light chain variable region (VL) comprising VL complementarity determining regions (CDRs) 1, 2, and 3; wherein the VH CDRs 1, 2, and 3, have the amino acid sequences set forth in SEQ ID NO:4, SEQ ID NO:5, and SEQ ID NO:6, respectively, or as set forth in SEQ ID NO:12, SEQ ID NO:13, and SEQ ID NO:14, respectively; and, wherein the VL CDRs 1, 2, and 3, have the amino acid sequences set forth in SEQ ID NO:8, SEQ ID NO:9, and SEQ ID NO:10, respectively, or as set forth in SEQ ID NO:16, SEQ ID NO:17, and SEQ ID NO:18, respectively.

The compound may be an antibody, or an antigen-binding portion thereof, having a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3, wherein the CDR1 region has the amino acid sequence of SEQ ID NO:4 or SEQ ID NO:12; wherein the CDR2 region has the amino acid sequence of SEQ ID NO:5 or SEQ ID NO:13; and wherein the CDR3 region has the amino acid sequence of SEQ ID NO:6 or SEQ ID NO:14. The compound may be an antibody, or an antigen-binding portion thereof, having a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3, wherein CDR 1, 2, and 3, have the amino acid sequences as set forth in SEQ ID NO:4, SEQ ID NO:5, and SEQ ID NO:6, respectively, or as set forth in SEQ ID NO:12, SEQ ID NO:13, and SEQ ID NO:14, respectively. The compound may be an antibody, or an antigen-binding portion thereof, having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3, wherein the CDR1 region has the amino acid sequence of SEQ ID NO:8 or SEQ ID NO:16; wherein the CDR2 region has the amino acid sequence of SEQ ID NO:9 or SEQ ID NO:17; and wherein the CDR3 region has the amino acid sequence of SEQ ID NO:10 or SEQ ID NO:18. The compound may be an antibody, or an antigen-binding portion thereof, having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3, wherein CDR 1, 2, and 3, have the amino acid sequences as set forth in SEQ ID NO:8, SEQ ID NO:9, and SEQ ID NO:10, respectively, or as set forth in SEQ ID NO:16, SEQ ID NO:17, and SEQ ID NO:18, respectively.

The compound may be an antibody, or an antigen-binding portion thereof, having: a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3; and, a light chain variable region (VL) having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3; wherein the VH CDR1 region has the amino acid sequence of SEQ ID NO:4 or SEQ ID NO:12; wherein the VH CDR2 region has the amino acid sequence of SEQ ID NO:5 or SEQ ID NO:13; wherein the VH CDR3 region has the amino acid sequence of SEQ ID NO:6 or SEQ ID NO:14; wherein the VL CDR1 region has the amino acid sequence of SEQ ID NO:8 or SEQ ID NO:16; wherein the VL CDR2 region has the amino acid sequence of SEQ ID NO:9 or SEQ ID NO:17; and, wherein the VL CDR3 region has the amino acid sequence of SEQ ID NO:10 of SEQ ID NO:18.

The compound may be an antibody, or an antigen-binding portion thereof, having: a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3; and, a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3; wherein the VH CDRs 1, 2, and 3 have the amino acid sequences set forth in SEQ ID NO:4, SEQ ID NO:5, and SEQ ID NO:6, respectively, or as set forth in SEQ ID NO:12, SEQ ID NO:13, and SEQ ID NO:14, respectively; and, wherein the VL CDRs 1, 2, and 3, have the amino acid sequences set forth in SEQ ID NO:8, SEQ ID NO:9, and SEQ ID NO:10, respectively, or as set forth in SEQ ID NO:16, SEQ ID NO:17, and SEQ ID NO:18, respectively.

The compound may be an antibody, or an antigen-binding portion thereof, that comprises: a heavy chain variable region (VH) comprising VH complementarity determining regions (CDRs) 1, 2, and 3; and, a light chain variable region (VL) comprising VL complementarity determining regions (CDRs) 1, 2, and 3; wherein the VH CDRs 1, 2, and 3, have the amino acid sequences set forth in SEQ ID NO:4, SEQ ID NO:5, and SEQ ID NO:6, respectively; and, wherein the VL CDRs 1, 2, and 3, have the amino acid sequences set forth in SEQ ID NO:8, SEQ ID NO:9, and SEQ ID NO:10, respectively.

The compound may be an antibody, or an antigen-binding portion thereof, that comprises: a heavy chain variable region (VH) comprising VH complementarity determining regions (CDRs) 1, 2, and 3; and, a light chain variable region (VL) comprising VL complementarity determining regions (CDRs) 1, 2, and 3; wherein the VH CDRs 1, 2, and 3 have the amino acid sequences set forth in SEQ ID NO:12, SEQ ID NO:13, and SEQ ID NO:14, respectively; and, wherein the VL CDRs 1, 2, and 3, have the amino acid sequences set forth in SEQ ID NO:16, SEQ ID NO:17, and SEQ ID NO:18, respectively.

The compound may be an antibody selected from W6/32 monoclonal antibody and DX17 monoclonal antibody.

One aspect provides a compound of the disclosure for use in activating a human immune cell expressing a LILRB protein, in a human; wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a TCR.

One aspect provides a compound of the disclosure for use in treating cancer, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in treating an infectious disease, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in converting a Th2-mediated immune response in an individual into a Th1-mediated immune response, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in treating a Th2-mediated immune response in an individual, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in treating a Th2-mediated immune response to an allergen in an individual, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in preventing a Th2-mediated immune reaction to an allergen in an individual, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

One aspect provides a compound of the disclosure for use in augmenting the immunogenicity of a vaccine in an individual, wherein binding of the compound to the binding site on an MHC class I HLA molecule inhibits interaction of the MHC class I HLA molecule with the LILRB protein; and, wherein binding of the compound to the binding site on the MHC class I HLA molecule does not affect the interaction of the MHC class I HLA molecule with a T-cell receptor (TCR).

### BRIEF DESCRIPTION OF THE DRAWINGS

FIG. 1 provides a comparison of the interactions of Ly-49, KIR, and LILRB with MHC-I. Mouse Ly-49 binds to the $32m site which is distinct from the TCR binding site. Human KIR3DL1 binds to HLA at a site overlapping with the TCR binding site and distant from the β2m site. Human LILRB binds to HLA at the β2m binding site.

FIG. 2 provides a depiction of the LILR family of molecules. LILRA's are activating receptors, while LILRB's are inhibitory receptors. LILRs are currently known to be present in monocytes, macrophages, dendritic cells, NK cells, granulocytes B-cell, T-cells, hematopoietic stem cells, tumor cells, and neurons. Ligands for LILR molecule include MHC-1, viruses (CMV, Dengue), bacteria (E. coli, S. aureus, H. pylori), bone marrow derived antigen-2, angiopoietin, Nogo receptor inhibitors, and β-amyloid protein.

FIG. 3 depicts the results from surface plasmon resonance analysis of the binding of W6/32. The figure shows that W6/32 only binds to the complex of human MHC-I heavy chain and human β2m (light chain).

FIG. 4 shows that the binding of an LILRB1 tetramer is blocked by both W6/32 and DX17. Total PBMC or gated monocytes or lymphocytes were analyzed after being incubated with PE-labeled LILRB1 in the presence or DX-17 antibody, W6/32 antibody, or a control mAb.

FIG. 5 Comparison of the crystal structure of DX17 complexed with HLA-B4405, and the published crystal structure of HLA and LILRB1.

FIG. 6 depicts the interactions between amino acid residues in the heavy and light chains of DX17, and the heavy and light chains of HLA, as determined from analysis of the crystal structure.

FIGS. 7A-7D further depict interactions between HLA-B4405 and DX17 monoclonal antibody. FIG. 7A shows which amino acid residues from HLA-B4405 heavy chain interact with amino acids residues in the DX17 antibody heavy chain. FIG. 7B shows which amino acid residues from HLA-B4405 beta-2 microglobulin interact with amino acids residues in the DX17 antibody heavy chain. FIG. 7C shows which amino acid residues from HLA-B4405 heavy chain interact with amino acids residues in the DX17 antibody light chain. FIG. 7D shows which amino acid residues from HLA-B4405 beta-2 microglobulin interact with amino acids residues in the DX17 antibody light chain. Interacting amino acid residues are underlined and bolded.

FIGS. 8A-8C demonstrate activation of human NK cells after 4 days of culture in the presence of W6/32 Fab. Human PBMC were cultured in the presence of isotype control mAb or W6/32 Fab for four days. No stimuli were added. In the presence of W6/32, marked expansion of CD16+NK cells was observed (FIG. 8A), the CD16+ cells had proliferated based on uptake of Ki-67(FIG. 8B), and produced enhanced amounts of IFN-gamma (FIG. 8C).

FIG. 9 demonstrates that neither W6/32 or DX17 block TCR antigen recognition. Jurkat T cells were transfected with a TCR specific for the GP100 tumor antigen and then stained with an HLA-A2/GP100 tetramer in the presence of the indicated reagents. Binding of the tetramer was blocked by the anti-HLA-A2 mAb, but not by W6/32 or DX17.

FIGS. 10A-D depict the results of a study showing the effects of W6/32 proliferation of NK, CD4+ and CD8+ cells in mice. FIG. 10A) shows timeline of study. NSG mice were reconstituted with human PBMC and two days later treated with W6/32 or isotype control. Four days later proliferation of human NK cells, CD4+ and CD8+ cells was observed in the spleen (FIG. 10B), liver (FIG. 10C) and lungs (FIG. 10D) as quantitated by Ki-67 staining. (Ki-67 labels cells which have proliferated in the previous 48 hours).

FIGS. 11A & B show that W6/32 enhances immune responses in IL-15 NOG mice. FIG. 11A) timeline of study. NOG mice (similar to NSG) expressing a transgene encoding IL-15 (which promotes the survival of NK cells) were injected with a preparation of human lymphocytes enriched for NK cells. On day 14 after engraftment, mice were treated with W6/32 and human cells were analyzed in the liver 48 hours later. FIG. 11B) Marked enhancement of the proliferation of NK cells (CD16+), CD4+ and CD8+ cells, was observed.

FIGS. 12A-12D show that the Fc fragment of intact mAbs prevent NK cell activation by interacting with an inhibitory Fc receptor on the NK cell. DX17 LALA PG is a chimeric antibody having a human IgG1 Fc containing two mutations, LALA & PG, that prevent binding to human Fc receptors. FIG. 12A shows that the chimeric antibody was active and inhibited binding of W6/32 to PBMC. FIG. 12B shows that chimeric DX17, but not wild-type DX17, induced clumping of human PBMC in culture. FIGS. 12C & 12D show that chimeric DX17, but not wild-type DX17, induced NK cell proliferation as measured by Ki-67 expression (FIG. 12C) and dilution of CellTrace™ Violet (CTV) (FIG. 12D).

FIGS. 13A & 13B show that W6/32 induces tumor immunity.

FIG. 13A illustrates the experimental design for measuring the effect of W6/32 Fab on tumor size. FIG. 13B shows tumors removed from mice treated with either mIgG2a (Top row) or W6/32 Fab (Bottom row)

### DETAILED DESCRIPTION

The present disclosure generally involves a method of modulating the interaction of proteins of the immune system for the purpose of preventing or treating disease. More specifically, the disclosure relates to a method of using specific compounds to inhibit interactions between cell-surface expressed LILRB, and MHC class I HLA molecules. The method is based on the knowledge that during interaction of a cell expressing LILRB with a cell expressing MHC class I HLA molecules, LILRB binds to HLA, resulting in negative regulation of immune activation (i.e., suppression of immune activation). While such immunosuppression may be beneficial; for example, evasion of the maternal immune response by the semi-allogenic fetus during pregnancy, it can also result in an inability of the immune system to clear disease. For example, tumors and cancer cells that express MHC class I HLA molecules may avoid immune detection by preventing activation of immune cells via interaction of the tumor-expressed HLA molecule and the immune-cell expressed LILRB protein. The inventors have discovered that compounds that bind MHC class I HLA molecules and inhibit their interaction with LILRB proteins cause activation of the immune response. Such activation may be utilized to treat certain disease such as infectious diseases and cancer. Thus, the disclosure may generally be practiced by contacting an MHC class I HLA molecule with a compound that binds to the HLA molecule, thereby preventing it from interacting with a LIRLB protein. The result of preventing the interaction of the LILRB protein and MHC class I HLA molecule is that the LILRB-expressing cell does not receive a negative regulatory signal, and consequently, the LILRB-expressing cell is activated. Activation of the immunes response resulting from such inhibition of binding may be used to prevent, treat or alter a disease. Preferably, the compound does not affect the ability of MHC class I HLA molecule to interact with a TCR.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the claims.

It must be noted that as used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Certain features of the disclosure, which are described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

Publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

One aspect is a method comprising administering to a human subject a compound that recognizes a binding site on an MHC class I HLA molecule,
wherein binding of the compound to the binding site on MHC class I HLA molecule inhibits the interaction of HLA molecule with a LILRB protein; and,
wherein binding of the compound to the binding site on MHC class I HLA molecule does not affect interaction between the HLA molecule and a TCR.

For the purposes of this disclosure, the terms “MHC-1 HLA molecule”, “MHC class I HLA”, “class I HLA”, and “HLA molecule”, and the like, may be used interchangeably. The term “HLA” means human leukocyte antigen and refers to a heterodimer composed of a heavy a chain, and a smaller β chain (β2-microglobulin). MHC class I HLA molecules useful for practicing the disclosure include, but are not limited to, HLA-A, HLA-B, HLA-C, HLA-E, HLA-F, and HLA-G. Thus, in certain aspects, the MHC class I HLA may be HLA-A. In certain aspects, the MHC class I HLA may be HLA-B. In certain aspects, the MHC class I HLA may be HLA-C. In certain aspects, the MHC class I HLA may be HLA-E. In certain aspects, the MHC class I HLA may be HLA-F. In certain aspects, the MHC class I HLA may be HLA-G.

The term “LILR” means leukocyte immunoglobulin-like receptor” and refers to a family of receptors having 2-4 extracellular Ig-like domains, and that are characterized as either activating (A) or inhibitory (B). LILRB refers to a class of receptors that are inhibitory. That is, binding of an LILRB to MHC class I HLA molecule results in suppression of activation of the LILRB-expressing cell. The term LILRB encompasses the receptors LILRB1-LILRB5, which are all inhibitory receptors.

As used herein, the terms subject, individual, and the like, can be used interchangeably, and by themselves do not denote a particular age, sex, race, or the like. Thus, subjects of any age, whether male or female, are intended to be covered by the present disclosure. In certain aspects, the subject may be a human. Methods of the present disclosure can be applied to any race of human, including, for example, Caucasian (white), African-American (black), Native American, Native Hawaiian, Hispanic, Latino, Asian, and European. Such characteristics may be significant. In such cases, the significant characteristic(s) (e.g., age, sex, race, etc.) will be denoted. In certain aspects, the subject may be suffering from an infection or disease. In certain aspects, the subject may be suspected having an infection or disease. In certain aspects, the subject may be free of infection or disease.

The term “administering” means introducing the compound to the subject via any route that results in the compound coming in contact with an MHC class I HLA molecule in the subject. The compound may be administered by means including, but not limited to, traditional syringes, needleless injection devices, or microprojectile bombardment gene guns. Using such devices, suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the compound may be formulated in aqueous solutions, preferably in physiologically compatible buffers such as Hanks' solution, Ringer's solution, or physiological saline buffer. In certain aspects, the compound may be delivered orally using suitable means such as a pill, a capsule, or a solution.

As used herein, the term“compound” refers to any type of molecule that specifically binds to a binding site on an HLA molecule. A compound can be a peptide, a protein (e.g., an antibody), a DNA or RNA molecule (e.g., an aptamer), a small molecule (e.g., an organic molecule), or any combination thereof. The compound may be isolated (i.e., purified) from a natural source, or it may be produced recombinantly, synthetically, or produced using any combination of such techniques. In certain aspects, the compound is a small molecule. In certain aspects, the compound is a peptide. In certain aspects, the compound is a nucleic acid molecule comprising RNA and/or DNA, such as an aptamer. In certain aspects, the compound comprises a protein. In certain aspects, the compound may be an antibody.

Binding of the compound to an MHC class HLA molecule refers to non-covalent, physical or chemical interactions between the compound and the HLA molecule, and includes ionic, non-ionic, van der waals, hydrophobic interactions, and the like. The term “specifically binds”, “binds specifically to”, and the like, mean that the HLA-binding portion of the compound binds the HLA molecule with an affinity significantly greater than the affinity of the HLA-binding portion of the compound for a molecule unrelated to HLA

Preferred compounds are those that bind HLA-A, HLA-B, HLA-C, HLA-D, HLA-E, HLA-F, and HLA-G. Such compounds are beneficial because blocking the binding site on MHC for all LILRBs results in greater enhancement of the activation of the immune response, and hence a greater therapeutic response.

The phrase “binding site on HLA molecule”, and the like, refers to the group of amino acid residues in the MHC class I HLA molecule heavy chain and/or β2-microglobuin proteins that interact (ionic bonds, non-ionic bonds, etc.) with the compound. The amino acid residues that form the binding site on MHC class I HLA molecule may be immediately adjacent to one another within a HLA heavy chain or β2-microglobulin protein. Alternatively, the amino acid residues that form the binding site on the MHC class I HLA molecule may be from distant parts of the heavy chain and/or the β2-microglobulin protein. In such a scenario, folding of the MHC class I HLA molecule into its final conformation brings amino acid residues into special proximity to form the compound binding site. The binding site on the HLA molecule may also be formed by amino acid residues present in both the MHC class I HLA heavy chain protein and the MHC class I HLA β2-microglobulin protein.

In certain aspects, the binding site on the MHC class I HLA molecule comprises one or more amino acid residues corresponding to one or more amino acid residues selected from the group consisting of Q87 of SEQ ID NO:1, K121 of SEQ ID NO:1, D122 of SEQ ID NO:1, A136 of SEQ ID NO:1, D137 of SEQ ID NO:1, T225 of SEQ ID NO:1, Q226 of SEQ ID NO:1, D227 of SEQ ID NO:1, T228 of SEQ ID NO:1, E232 of SEQ ID NO:1, M0 of SEQ ID NO:2, 11 of SEQ ID NO:2, Q2 of SEQ ID NO:2, R3 of SEQ ID NO:2, K6 of SEQ ID NO:2, K58 of SEQ ID NO:2, D59 of SEQ ID NO:2, and W60 of SEQ ID NO:2. SEQ ID NO:2 is the sequence of HLAB4405 β-2 microglobulin. For the present studies, the protein was expressed in bacteria, which required an N-terminal methionine. In order to preserve the canonical numbering for β-2 microglobulin, the M was numbered “0”. Thus, while the adjoined isoleucine is the second amino acid in SEQ ID NO:2, it is referred to as 11, in keeping with the canonical numbering system. Reference to a corresponding amino acid position recognizes the fact that the sequences of closely related MHC class I HLA proteins (i.e., the heavy a chain or the β-2 microglobulin protein) might not align exactly over the entire length of the protein. For example, due to allelic variation (e.g., insertions or deletions in a heavy a chain or the β-2 microglobulin protein) the D112 contact residue, which is in amino acid position number 122 (starting from the N-terminal) in MHC class I HLA B4405, might be in position 121 or 123 in a second, closely related MHC class I HLA heavy a chain protein. It should be understood that while the D residue might be shifted in position in the second MHC class I HLA heavy a chain protein, it would still perform the same function (e.g., serve as a contact residue for binding of the antibody), and would still be in the same relative spatial orientation with the other contact residues, as is observed in the MHC class I HLA B4405 heavy a chain protein.

In certain aspects, the binding site on the MHC class I HLA molecule comprises one or more amino acid residues selected from the group consisting of:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

In certain aspects, the binding site on the MHC class I HLA molecule comprises at least 2, at least 3, at least 4, at least 5, at least 6, at least 7, at least 8, at least 9, at least 10, at least 11, at least 12, at least 13, at least 14, at least 15, at least 16, at least 17, or at least 18, amino acid residues selected from the group consisting of:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.
In certain aspects, the binding site on the MHC class I HLA molecule comprises:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

As used herein, the phrases, “inhibit(s) the interaction of an HLA molecule with a LILRB protein”, “block the interaction of an HLA molecule with LILRB”, “modulate the interaction of HLA with LILRB”, and the like, mean that binding of the compound to an MHC class I HLA molecule reduces the strength of binding between the MHC class I HLA molecule and a LILRB protein, or completely or partially prevents interaction of the MHC class I HLA molecule and the LILRB protein, thereby reducing or preventing the immunoinhibitory signal between the MHC class I HLA molecule and a LILRB protein. The term “immunoinhibitory signal” refers to the interaction between an MHC class I HLA molecule and an LILRB protein that reduces or prevents the immunoactivity of a LILRB-bearing immune cell against an HLA-expressing cell (a.k.a., target cell). According to the present disclosure, inhibition of the interaction between the MHC class I HLA molecule and a LILRB protein results in the ability of a cell expressing the LILRB protein to increase specific lysis of the target cell to more than about 20%, preferably at least about 30%, at least about 40%, at least about 50%, at least about 100%, at least about 200%, or at least about 500% of the specific lysis obtained at the same effector:target cell ratio with LILRB-expressing cells that are not blocked from interacting with the MHC class I HLA molecule.

In the present disclosure, binding of the compound to an MHC class I HLA molecule does not significantly affect the interaction between the MHC class I HLA molecule and a TCR. That is, binding of the compound to the MHC class I HLA molecule does not inhibit the MHC class I HLA molecule from presenting a bound peptide to the TCR. As used herein, the term “T-cell receptor” (TCR), refers to all types, and isotypes, of T cells, including, but not limited to, cytotoxic T cells 9CD8+, helper T cells (CD4+), regulatory T cells, alpha:beta (αβ) T cells, and gamma:delta (γδ) T cells. With regard to T cells, the term “significantly” means that the strength of interaction between the MHC class I HLA containing a peptide and a TCR in the presence or absence of the compound differs by less than about 20%, less than about 10%, or less than about 5%.

In certain aspects, the compound may be an antibody. The term “antibody” refers to a molecule in which the structure and/or function is/are based on the structure and/or function of a partial, full-length or whole immunoglobulin molecule. As used herein, the term “antibody” includes monoclonal antibodies (mAbs), chimeric antibodies, single chain antibodies, human antibodies, humanized antibodies, as well as antibody fragments, such as Fab fragments, and derivatives. The term also includes antibodies in which CDR sequences derived from the germline of one mammalian species, such as a mouse, have been grafted onto the framework sequences of a second mammalian species, such as a primate (e.g., human primate).

Antibody fragments or derivatives further comprise F(ab′)2, Fv, scFv fragments or single domain antibodies such as domain antibodies or nanobodies, single variable domain antibodies or immunoglobulin single variable domain comprising merely one variable domain, which might be VHH, VH or VL, that specifically bind an antigen or epitope independently of other V regions or domains. An immunoglobulin single variable domain encompasses not only an isolated antibody single variable domain polypeptide, but also larger polypeptides that comprise one or more monomers of an antibody single variable domain polypeptide sequence.

In aspects of the disclosure in which the compound is an antibody, the antibody may not be capable of being bound by, and may not be capable of binding, an Fc receptor. Thus, the antibody may comprise one or more mutations that result in the antibody being incapable of being bound by, and incapable of binding, an Fc receptor. Such mutations are known in the art and include, but are not limited to, substitution of the leucine corresponding to Leu 234, substitution of the leucine corresponding to Leu 235, and/or substitution of the proline corresponding to Pro 329 of an IgG antibody. In certain aspects, the IgG antibody may be of the isotype IgG1 κ. In certain aspects, an antibody used in methods of the disclosure may comprise substitution of the leucine corresponding to Leu234, substitution of the leucine corresponding to Leu235, and/or substitution of the proline corresponding to Pro329 of an IgG antibody, which may be an IgG antibody of isotype IgG1 κ. In certain aspects, the substituting amino acid may be any amino acid residue that allows the antibody to inhibit interaction of the MHC class I HLA with an LILRB protein. In certain aspects, substitution of the leucine corresponding to Leu 234, and/or substitution of the leucine corresponding to Leu 235 may comprise substitution with an alanine. In certain aspects, substitution of the proline corresponding to Pro 329 may comprise substitution with a glycine.

In certain aspects, the compound may be an antibody, or a fragment or derivative thereof, which may be an IgA immunoglobulin, an IgD immunoglobulin, an IgE immunoglobulin, an IgG immunoglobulin, or an IgM immunoglobulin. In certain aspects, the compound may be a mAb, a chimeric antibody, a single chain antibody, a human antibody, a humanized antibody, an antibody fragment, a Fab, a F(ab′)2, an Fv region, a scFv fragment or a single domain antibody.

The binding site for the antibody on the MHC class I HLA molecule may be at a location other than the location where the LILRB protein interacts with the HLA molecule. In this regard, binding of the antibody may cause structural changes in the MHC class I HLA molecule (e.g., allosteric changes), such that binding of the LILRB protein is reduced or prevented. The binding site for the antibody on the MHC class I HLA molecule may be at a location that overlaps with the location where the LILRB protein interacts with the HLA molecule. Overlapping means once bound to the HLA molecule, the compound sterically inhibits interaction of the LILRB protein with the MHC class I HLA molecule. In such a scenario, the binding site may, but need not, share one or more amino acid residues with the HLA amino acid residues that interact with the LILRB protein.

W6/32 and DX17 are currently available monoclonal antibodies that are known to bind to an MHC class I HLA molecule (e.g., ThermoFisher Catalog #14-9983-82, and BD Biosciences Catalog #560168, respectively). The inventors have discovered that the W6/32 monoclonal antibody and the DX17 monoclonal antibody are able to inhibit interaction between a LILRB protein and an MHC class I HLA molecule without significantly affecting interaction between the MCH class I HLA molecule and a TCR. Thus, in certain aspects, the compound of the disclosure may be an antibody, and binding of the antibody to an MHC class I HLA molecule inhibits binding of the W6/32 monoclonal antibody or the DX17 monoclonal antibody to the same MHC class I HLA molecule. The binding site for the antibody may be at a location other than the location where the W6/32 monoclonal antibody, or the DX17 monoclonal antibody, binds the MHC class I HLA molecule. The binding site for the antibody may be at a location that overlaps where the W6/32 monoclonal antibody, or the DX17 monoclonal antibody, binds the MHC class I HLA molecule. The binding site for the antibody may, but need not, share one or more amino acid residues with the MHC class I HLA amino acid residues that contact with the W6/32 monoclonal antibody or the DX17 monoclonal antibody.

In certain aspects, the antibody may bind to HLA-A, HLA-B, HLA-C, HLA-D, HLA-E, HLA-F, and HLA-G. Such an antibody is preferred because blocking the binding site on MHC for all LILRBs results in greater enhancement of the activation of the immune response, and hence a greater therapeutic response.

In certain aspects, the antibody may bind a site on the MHC class I HLA molecule comprising one or more amino acid residues corresponding to one or more amino acid residues selected from the group consisting of Q87 of SEQ ID NO:1, K121 of SEQ ID NO:1, D122 of SEQ ID NO:1, A136 of SEQ ID NO:1, D137 of SEQ ID NO:1, T225 of SEQ ID NO:1, Q226 of SEQ ID NO:1, D227 of SEQ ID NO:1, T228 of SEQ ID NO:1, E232 of SEQ ID NO:1, M0 of SEQ ID NO:2, 11 of SEQ ID NO:2, Q2 of SEQ ID NO:2, R3 of SEQ ID NO:2, K6 of SEQ ID NO:2, K58 of SEQ ID NO:2, D59 of SEQ ID NO:2, and W60 of SEQ ID NO:2. Reference to a corresponding amino acid position recognizes the fact that the sequences of closely related MHC class I HLA proteins (i.e., the heavy a chain or the β-2 microglobulin protein) might not align exactly over the entire length of the protein. For example, due to allelic variation (e.g., insertions or deletions in a heavy a chain or the β-2 microglobulin protein) the D112 contact residue, which is in amino acid position number 122 (starting from the N-terminal) in HLA B4405, might be in position 121 or 123 in a second, closely related HLA heavy α chain protein. It should be understood that while the D residue might be shifted in position in the second HLA heavy α chain protein, it would still perform the same function (e.g., serve as a contact residue for binding of the antibody), and would still be in the same relative spatial orientation with the other contact residues, as is observed in the HLA B4405 heavy α chain protein. In certain aspects, the antibody may bind a site on the MHC class I HLA molecule comprising one or more amino acid residues selected from the group consisting of:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

In certain aspects, the antibody may bind a site on the HLA molecule comprising at least 2, at least 3, at least 4, at least 5, at least 6, at least 7, at least 8, at least 9, at least 10, at least 11, at least 12, at least 13, at least 14, at least 15, at least 16, at least 17, or at least 18, amino acid residues selected from the group consisting of:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.
In certain aspects, the antibody may bind a site on the HLA molecule comprising:
a glutamine at a position corresponding to amino acid position 87 of SEQ ID NO:1;
a lysine at a position corresponding 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

In certain aspects, the compound may be an antibody, or an antigen-binding portion thereof, that comprises a heavy chain variable region (VH) comprising VH complementarity determining regions (CDRs) 1, 2, and 3, wherein the CDR1 region has the amino acid sequence of SEQ ID NO:4 or SEQ ID NO:12; wherein the CDR2 region has the amino acid sequence of SEQ ID NO:5 or SEQ ID NO:13; and wherein the CDR3 region has the amino acid sequence of SEQ ID NO:6 or SEQ ID NO:14.

In certain aspects, the compound may be an antibody, or an antigen-binding portion thereof, that comprises a heavy chain variable region (VH) comprising VH complementarity determining regions (CDRs) 1, 2, and 3, having an amino acid sequences as set forth in SEQ ID NO:4, SEQ ID NO:5, and SEQ ID NO:6, respectively, or as set forth in SEQ ID NO:12, SEQ ID NO:13, and SEQ ID NO:14, respectively.

In certain aspects, the compound may be an antibody, or an antigen-binding portion thereof, that comprises a light chain variable region (VL) comprising VL complementarity determining regions (CDRs) 1, 2, and 3, wherein the CDR1 region has the amino acid sequence of SEQ ID NO:8 or SEQ ID NO:16; wherein the CDR2 region has the amino acid sequence of SEQ ID NO:9 or SEQ ID NO:17; and wherein the CDR3 region has the amino acid sequence of SEQ ID NO:10 or SEQ ID NO:18.

In certain aspects, the compound may be an antibody, or an antigen-binding portion thereof, that comprises a light chain variable region (VL) comprising VL complementarity determining regions (CDRs) 1, 2, and 3, having the amino acid sequences set forth in SEQ ID NO:8, SEQ ID NO:9, and SEQ ID NO:10, respectively, or in SEQ ID NO:16, SEQ ID NO:17, and SEQ ID NO:18, respectively.

In certain aspects, the compound may be an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDR1 region has the amino acid sequence of SEQ ID NO:4 or SEQ ID NO:12;
wherein the VH CDR2 region has the amino acid sequence of SEQ ID NO:5 or SEQ ID NO:13;
wherein the VH CDR3 region has the amino acid sequence of SEQ ID NO:6 or SEQ ID NO:14;
wherein the VL CDR1 region has the amino acid sequence of SEQ ID NO:8 or SEQ ID NO:16;
wherein the VL CDR2 region has the amino acid sequence of SEQ ID NO:9 or SEQ ID NO:17; and,
wherein the VL CDR3 region has the amino acid sequence of SEQ ID NO:10 or SEQ ID NO:18.

In certain aspects, the compound may be an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDRs 1, 2, and 3 have the amino acid sequences set forth in SEQ ID NO:4, SEQ ID NO:5, and SEQ ID NO:6, respectively, or in SEQ ID NO:12, SEQ ID NO:13, and SEQ ID NO:14, respectively; and,
wherein the VL CDRs 1, 2, and 3, have the amino acid sequences set forth in SEQ ID NO:8, SEQ ID NO:9, and SEQ ID NO:10, respectively, or in SEQ ID NO:16, SEQ ID NO:17, and SEQ ID NO:18, respectively.

In certain aspects, the compound may be an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDRs 1, 2, and 3 have the amino acid sequences set forth in SEQ ID NO:4, SEQ ID NO:5, and SEQ ID NO:6, respectively; and,
wherein the VL CDRs 1, 2, and 3, have the amino acid sequences set forth in SEQ ID NO:8, SEQ ID NO:9, and SEQ ID NO:10, respectively.

In certain aspects, the compound may be an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDRs 1, 2, and 3 have the amino acid sequences set forth in SEQ ID NO:12, SEQ ID NO:13, and SEQ ID NO:14, respectively; and,
wherein the VL CDRs 1, 2, and 3, have the amino acid sequences set forth in SEQ ID NO:16, SEQ ID NO:17, and SEQ ID NO:18, respectively.

In certain aspects, the compound may be an antibody, or an antigen-binding portion thereof, having a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3, wherein the CDR1 region has the amino acid sequence of SEQ ID NO:4 or SEQ ID NO:12; wherein the CDR2 region has the amino acid sequence of SEQ ID NO:5 or SEQ ID NO:13; and wherein the CDR3 region has the amino acid sequence of SEQ ID NO:6 or SEQ ID NO:14.

In certain aspects, the compound may be an antibody, or an antigen-binding portion thereof, having a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3, wherein CDR 1, 2, and 3, have the amino acid sequences as set forth in SEQ ID NO:4, SEQ ID NO:5, and SEQ ID NO:6, respectively, or as set forth in SEQ ID NO:12, SEQ ID NO:13, and SEQ ID NO:14, respectively.

In certain aspects, the compound may be an antibody, or an antigen-binding portion thereof, having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3, wherein the CDR1 region has the amino acid sequence of SEQ ID NO:8 or SEQ ID NO:16; wherein the CDR2 region has the amino acid sequence of SEQ ID NO:9 or SEQ ID NO:17; and wherein the CDR3 region has the amino acid sequence of SEQ ID NO:10 or SEQ ID NO:18.

In certain aspects, the compound may be an antibody, or an antigen-binding portion thereof, having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3, wherein CDR 1, 2, and 3, have the amino acid sequences as set forth in SEQ ID NO:8, SEQ ID NO:9, and SEQ ID NO:10, respectively, or as set forth in SEQ ID NO:16, SEQ ID NO:17, and SEQ ID NO:18, respectively.

In certain aspects, the compound may be an antibody, or an antigen-binding portion thereof, having a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3; and,
a light chain variable region (VL) having a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3;
   wherein the VH CDR1 region has the amino acid sequence of SEQ ID NO:4 or SEQ ID NO:12;
   wherein the VH CDR2 region has the amino acid sequence of SEQ ID NO:5 or SEQ ID NO:13;
   wherein the VH CDR3 region has the amino acid sequence of SEQ ID NO:6 or SEQ ID NO:14;
   wherein the VL CDR1 region has the amino acid sequence of SEQ ID NO:8 or SEQ ID NO:16;
   wherein the VL CDR2 region has the amino acid sequence of SEQ ID NO:9 or SEQ ID NO:17; and,
   wherein the VL CDR3 region has the amino acid sequence of SEQ ID NO:10 of SEQ ID NO:18.

In certain aspects, the compound may be an antibody, or an antigen-binding portion thereof, having:
a heavy chain variable region (VH) comprising an amino acid sequence at least 80%, at least 85%, at least 90%, at least 95%, at least 97%, or 100% identical to SEQ ID NO:3 or SEQ ID NO:11, and comprising complementarity determining regions (CDRs) 1, 2, and 3; and,
a light chain variable region (VL) comprising an amino acid sequence at least 80%, at least 85%, at least 90%, at least 95%, at least 97%, or 100% identical to SEQ ID NO:7 or SEQ ID NO:15, and having VL complementarity determining regions (CDRs) 1, 2, and 3;
wherein the VH CDRs 1, 2, and 3 have the amino acid sequences set forth in SEQ ID NO:4, SEQ ID NO:5, and SEQ ID NO:6, respectively, or in SEQ ID NO:12, SEQ ID NO:13, and SEQ ID NO:14, respectively; and,
wherein the VL CDRs 1, 2, and 3, have the amino acid sequences set forth in SEQ ID NO:8, SEQ ID NO:9, and SEQ ID NO:10, respectively, or in SEQ ID NO:16, SEQ ID NO:17, and SEQ ID NO:18, respectively.

In certain aspect, the compound may be an antibody selected from the group consisting of W6/32 monoclonal antibody and DX17 monoclonal antibody.

One embodiment is a method of preventing a LILRB protein from binding to an MHC class I HLA molecule, comprising contacting the MHC class I HLA molecule with a compound that recognizes a binding site on the HLA molecule;
wherein binding of the compound to the binding site on the MHC class I HLA molecule inhibits interaction of the HLA molecule with the LILRB protein; and,
wherein binding of the compound to the binding site on the MHC class I HLA molecule does not affect interaction between the HLA molecule and a TCR.

In certain aspects, the LILRB protein and the MHC class I HLA molecule may be present in an individual, and the compound is administered to the individual using a route of administration that allows the compound to contact the MHC class I HLA molecule. Suitable routes of administration include, but are not limited to, oral delivery or parenteral delivery, such as intramuscular, intradermal, subcutaneous, intramedullary injections, as well as, intrathecal, direct intraventricular, intravenous, intraperitoneal, intranasal, or intraocular injections.

In certain aspects, the method may be performed in vitro. That is, the LILRB protein and the MHC class I HLA molecule are not present within an intact organism (e.g., a mammal, a human, a mouse, etc.). In such a method, the LILRB protein and the MHC class I HLA molecule may be present in a vessel or container, such as, a vial, a tube (e.g., a microcentrifuge tube), a test tube, or the well of a cell culture plate. The LILRB protein and/or the MHC class I HLA molecule may or may not be present in a cell within the vessel. The LILRB protein and/or the MHC class I HLA molecule may be in a soluble form within the vessel. The LILRB protein and/or the MHC class I HLA molecule may be immobilized on the vessel wall. Further, if the LILRB protein and/or the MHC class I HLA molecule are present on cells in the vessel, such cells may be in a soluble form or they may be immobilized on the vessel wall.

One embodiment is a method of activating a human immune cell that expresses a LILRB protein, comprising blocking the interaction of the immune cell and an MHC class I HLA molecule by contacting the MHC class I molecule with a compound that recognizes a binding site on the MHC class I HLA molecule;
wherein binding of the compound to the binding site on the MHC class I HLA molecule inhibits interaction of the HLA molecule with the LILRB protein; and,
wherein binding of the compound to the binding site on the MHC class I HLA molecule does not affect interaction between the class I HLA molecule and a TCR.

In certain aspects, the method may be performed in vitro. That is, the LILRB protein and the MHC class I HLA molecule are not present within an intact organism (e.g., a mammal, a human, a mouse, etc.). In such a method, the LILRB protein and the MHC class I HLA molecule are present in a vessel or container, such as, a vial, a tube (e.g., a microcentrifuge tube), a test tube, or the well of a cell culture plate. The LILRB protein and/or the MHC class I HLA molecule may or may not be present in a cell within the vessel. The LILRB protein and/or the MHC class I HLA molecule may be in a soluble form within the vessel. The LILRB protein and/or the MHC class I HLA molecule may be immobilized on the vessel wall. Likewise, if the LILRB protein and/or the MHC class I HLA molecule are present on cells in the vessel, such cells may be in a soluble form of they may be immobilized on the vessel wall.

In certain aspects, the LILRB protein and the MHC class I HLA molecule may be in an individual, and the compound is administered to the individual using a route of administration that allows the compound to contact the MHC class I HLA molecule. Suitable routes of administration include, but are not limited to, oral delivery or parenteral delivery, such as intramuscular, intradermal, subcutaneous, intramedullary injections, as well as, intrathecal, direct intraventricular, intravenous, intraperitoneal, intranasal, or intraocular injections.

One embodiment is a method of treating a subject for a Th2-mediated reaction to an allergen, comprising administering to the individual a compound that recognizes a binding site on an MCH-I class I HLA molecule;
wherein binding of the compound to the binding site on the MHC class I HLA molecule inhibits interaction of the HLA molecule with a LILRB protein; and,
wherein binding of the compound to the binding site on the MHC class I HLA molecule does not affect the interaction between the HLA molecule and a TCR.

One embodiment is a method of preventing a Th2-mediated reaction to an allergen in an individual, comprising administering to the individual a compound that recognizes a binding site on an MHC class I HLA molecule;
wherein binding of the compound to the binding site on the MHC class I HLA molecule inhibits interaction of the HLA molecule with a LILRB protein;
wherein binding of the compound to the binding site on the MHC class I HLA molecule does not affect the interaction between the HLA molecule and a TCR; and,
wherein the compound is administered to the individual prior to the individual contacting the allergen.

As used herein, the term “allergen” refers to any substance that can cause a vigorous Th-2-type immune response (e.g., an allergic reaction). Examples of allergens include, but are not limited to, animal proteins (e.g., FelD1), dust, drugs (e.g., penicillin, sulfonamides, salicylates), food (e.g., eggs, nuts, milk, soy, fish, wheat, gluten, strawberries, corn, legumes, milk, sesame), fungal spores, insect and mites feces, cockroach calyx, insect venom, natural rubber, pollen, and metals (nickel, chromium).

The phrase “activating an immune cell”, “activating a human immune cell”, and the like, refers to a change in the activity of an immune cell in response to a stimulus, such that the cell begins to synthesize and/or release products that participate in processes that remove the stimulus. Such products include, for example, reactive oxygen species, enzymes (perforins, granzymes, etc.), cytokines, chemokines, and antibodies. For example, natural killer (NK) cells contain granules comprising perforins and granzymes. Upon activation of NK cells (e.g., by a target cell), perforin and granzyme are release from the cell, which results in inducing lysis and/or apoptosis of the target cell. Activated NK cells also express pattern recognition proteins (PRRs) (e.g., toll-like receptor (TLR)-2, -3, -4, -5, -7 and -8), and secrete interferon (IFN)-γ, tumor necrosis factor (TNF)-α, interleukin (IL)-5, IL-10, IL-13, and chemokines.

The compound may be administered to the individual using a route of administration that allows the compound to contact the MHC class I HLA molecule. Suitable routes of administration include, but are not limited to, oral delivery or parenteral delivery, such as intramuscular, intradermal, subcutaneous, intramedullary injections, as well as, intrathecal, direct intraventricular, intravenous, intraperitoneal, intranasal, or intraocular injections.

One embodiment is method of treating cancer in subject, comprising administering to the individual a compound that recognizes a binding site on an MHC class I HLA molecule;
wherein binding of the compound to the binding site on the MHC class I HLA molecule inhibits interaction of the HLA molecule with a LILRB protein; and,
wherein binding of the compound to the binding site on the MHC class I HLA molecule does not affect the interaction between the HLA molecule and a TCR.

In the context of the present disclosure, the term “cancer” refers to any neoplastic disorder, including, but not limited to, such cellular disorders as sarcoma, carcinoma, melanoma, leukemia, and lymphoma, which may include, but is not limited to, cancers in the breast, head and neck, ovaries, bladder, lung, pharynx, larynx, esophagus, stomach, small intestines, liver, pancreas, colon, female reproductive tract, male reproductive tract, prostate, kidneys and central nervous system.

In certain aspects of such embodiment, the method may comprise administering to the subject at least one additional therapeutic agent (e.g., drug). The at least one additional therapeutic agent may or may not be for treating the cancer. The at least one additional therapeutic agent may or may not be a checkpoint inhibitor. Examples of such checkpoint inhibitors include, but are not limited to, an anti-PD-1 antibody, an anti-PD-L1 an antibody, anti-CTLA-4 antibody, an anti-LAG-3 antibody, and an anti-TIM3 antibody. The at least one additional therapeutic agent may be chemotherapy, or a cytokine, examples of which include, but are not limited to, a cytokine that targets the IL-2/IL-2 receptor pathway, a cytokine that targets the IFNAR1/2 pathway, and granulocyte-macrophage colony-stimulating factor (GM-CSF).

The terms “treating”, “treatment”, and the like, refer to preventing, alleviating, managing, curing or reducing one or more symptoms or clinically relevant manifestations of a disease or disorder, unless contradicted by context. For example, “treating cancer” means administering a compound in order to eliminate or reduce the size of a tumor or reduce the number of cancer cells in a subject. “Treatment” of a patient in whom no symptoms or clinically relevant manifestations of a disease or disorder have been identified, for example, is preventive therapy, whereas “treatment” of a patient in whom symptoms or clinically relevant manifestations of a disease or disorder have been identified generally does not constitute preventive therapy. Nonetheless, it should be understood that the various therapeutic and prophylactic method and use facets of the disclosure are distinct from one in many respects (e.g., dosage of compound(s) to be delivered to a subject, timing of application, impetus for application, etc.) and may each be considered unique aspects of the disclosed methods.

One embodiment is method of treating an infectious disease in a subject, comprising administering to the individual a compound that recognizes a binding site on an MHC class I HLA molecule;
wherein binding of the compound to the binding site on the MHC class I HLA molecule inhibits interaction of the HLA molecule with a LILRB protein; and,
wherein binding of the compound to the binding site on the MHC class I HLA molecule does not affect the interaction between the HLA molecule and a TCR.

An infectious disease refers to a disease resulting from infection of the subject with a pathogen. In certain aspects the pathogen is an intracellular pathogen. The pathogen may be a virus, a bacterium, or a parasite. Examples of pathogens include, but are not limited to, Mycobacteria tuberculosis, Salmonella typhi, Salmonella typhimurium, Salmonella enteritidis, Listeria monocytogenes, Rickettsia rickettsia, Legionella pneumophila, Human Immunodeficiency Virus (HIV), herpesvirus, hepatitis virus, Varicella-Zoster virus, Epstein Barr virus, respiratory syncytial virus (RSV), papillomavirus, and coronavirus (including Middle Eastern Respiratory Syndrome virus (MERS), Severe Acute Respiratory Syndrome virus (SARS), and Severe Acute respiratory Syndrome coronavirus 2 (SARS-CoV-2).

T lymphocytes expressing the CD4 protein (a.k.a., helper T cells) can be classified as either Th1 or Th2, depending on the type of cytokines they produce. The cytokines produced by Th1 cells are known as Th1-type cytokines, while the cytokines produced by Th2 cells are known as Th2-type cytokines. Th1-type cytokines include IL-2, IL-12, IL-18, IL-27, and IFN-γ. Th2-type cytokines include IL-4, IL-5, and IL-13, and may also include IL-9, IL-10, IL-25, and amphiregulin. Generally, Th1-tend cytokines tend to produce proinflammatory immune responses, while Th2-type cytokines tend to produce anti-inflammatory immune responses. Redirecting a Th cell cytokine response from one type to another is a useful way to alleviate a Th-type imbalance and diseases related thereto. For example, allergy is generally regarded as a Th2-type weighted responses, and thus, redirecting a Th2-type immune response to a Th1-type response may be useful in treating allergy.

Thus, one embodiment is a method of converting a Th2-mediated immune response in a subject to a Th1-mediated immune response, comprising administering to the individual a compound that recognizes a binding site on an MHC class I HLA molecule;
wherein binding of the compound to the binding site on the MHC class I HLA molecule inhibits interaction of the HLA molecule with a LILRB protein; and,
wherein binding of the compound to the binding site on the MHC class I HLA molecule does not affect the interaction between the HLA molecule and a TCR.

One embodiment is method of treating a subject for a Th2-mediated disease, comprising administering to the individual a compound that recognizes a binding site on an MHC class I HLA molecule;
wherein binding of the compound to the binding site on the MHC class I HLA molecule inhibits interaction of the HLA molecule with a LILRB protein; and,
wherein binding of the compound to the binding site on the V molecule does not affect the interaction between the MHC class I HLA molecule and a TCR.

Examples of Th2-mediated disease include, but are not limited to, asthma, allergic rhinitis, atopic dermatitis, systemic progressive sclerosis, and Omenn's syndrome. In one aspect, the Th2-meditaed disease is selected form the group consisting of asthma, allergic rhinitis, atopic dermatitis, systemic progressive sclerosis, and Omenn's syndrome.

One embodiment is a method of augmenting the immunogenicity of a vaccine, comprising co-administering the vaccine and a compound that recognizes a binding site on an MHC class I HLA molecule;
wherein binding of the compound to the binding site on the MHC class I HLA molecule inhibits interaction of the HLA molecule with a LILRB protein; and,
wherein binding of the compound to the binding site on the MHC class I HLA molecule does not affect interaction between the MHC class I HLA molecule and a TCR.

As used herein, the term “augmenting the immune response”, “augment the immune response”, and the like, means enhancing or extending the duration of the immune response, or both. When referred to a property of an agent (e.g., the compound), the term “[able to] augment the immunogenicity” refers to the ability to enhance the immunogenicity of an antigen or vaccine, or the ability to extend the duration of the immune response to an antigen or vaccine, or both.

One aspect of the disclosure is a composition comprising a compound of the disclosure, for use in activating a human immune cell expressing a LILRB protein, treating cancer, treating an infectious disease, converting a Th2-mediated immune response to a Th1-mediated immune response, preventing or treating a Th2-mediated immune response, and/or augmenting the immunogenicity of a vaccine. In the use of such compositions, the compound binds to a binding site on an MHC class I HLA molecule and inhibits interaction of the MCH class I HLA molecule with the LILRB protein but does not affect the interaction of the MHC class I HLA molecule with a T-cell receptor.

One aspect of the disclosure is a kit comprising a composition of the disclosure. A kit of the disclosure may comprise additional components, such as vials, tubes, wipes, needles, syringes, bandages, and instructions for using the kit to activate an human immune cell expressing a LILRB protein, treat cancer, treat an infectious disease, convert a Th2-mediated immune response to a Th1-mediated immune response, prevent or treat a Th2-mediated immune response, and/or augment the immunogenicity of a vaccine.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Background

Mouse NK inhibitory receptors (Ly-49 antigens) are known to bind MHC Class I molecules, resulting in suppression of activation of the NK cell. Therefore, inhibition of the interaction of MHC-I (H-2 antigens in the mouse) with mouse NK Inhibitory receptors (Ly-49 antigens) should lead to a generalized activation of NK cells. To test this hypothesis, various mAbs that recognized murine H-2 were administered to mice, and the animals monitored for NK activation as indicated by proliferation and enhancement of cytokine production. It was found that pan anti-H-2 mAb (M1/42), when injected in normal mice over a 6-day period in the absence of other stimulation, broadly induced NK proliferation and enhancement of the production of interferon-g (IFN-g) and TNF-a. Treatment of mice with conventional anti-H-2 mAbs which recognized polymorphic determinants did not induce NK cell activation (J. Immunol. 205:567, 2020, attached). These animals also exhibited marked activation of memory phenotype CD4⁺ and CD8⁺ T cells and activation/proliferation of different antigen presenting cell populations including macrophages and dendritic cells.

Further analysis of these phenomena revealed that IFN-g and other cytokines induced IL-15 production by antigen presenting cells, which acted back on NK cells to enhance their activation and was also responsible for memory T cell phenotype activation. Thus, activation of the innate immune response by blocking Ly-49/H-2 interactions generated a complex loop which led to the activation of the adaptive immune response. Treatment with the M1/42 monoclonal antibody resulted in the clearance of chronic infection with clone 13 lymphocytic choriomeningitis virus and expansion of viral-antigen-specific CD8⁺ T cells. More importantly, treatment with the M1/42 monoclonal antibody markedly enhanced immunity to different transplantable tumors including one that is completely resistant to check point (PD-1) blockade. Further studies have demonstrated that M1/42 treatment dramatically inhibits tumor metastasis in well-established lung and liver models including a model of pancreatic cancer metastasis.

### Example 1. Characterization of the Properties of mAb M1/42

To define the binding site of mAb M1/42, mixed molecules were generated using the mouse H-2 heavy chain and human and/or mouse b₂ microglobulin (b₂m). mAb M1/42 only bound the heavy chain b2m complex composed of mouse b₂m and mouse heavy chain indicating that the binding site of the mAb was related not only to MHC-I heavy chain but also to b2m itself. These studies were also performed using blocking studies, FACS analysis, and surface plasmon resonance. As it has previously been shown that the binding site of mouse H-2 antigens for mouse Ly-49 antigens was also near the b2m binding site, this result was consistent with data showing that M1/42 blocked Ly-49/H-2 interactions. This result was confirmed by competitive binding studies on the FACS using recombinant Ly-49 molecules. Further research demonstrated that mAB M1/42 failed to inhibit antigen-specific T cell activation, consistent with the binding site of mAb M1/42 being at a site distinct from the peptide groove of the H-2 molecule. The activity of the mAb M1/42 was also not dependent on its Fc portion, as it induced the same magnitude NK activation in mice deficient in Fc receptors as in wild type mice. These results also indicate that the effects of mAb M1/42 are primarily due to blocking Ly-49/H-2 interactions and not secondary to signaling.

### Application in Humans

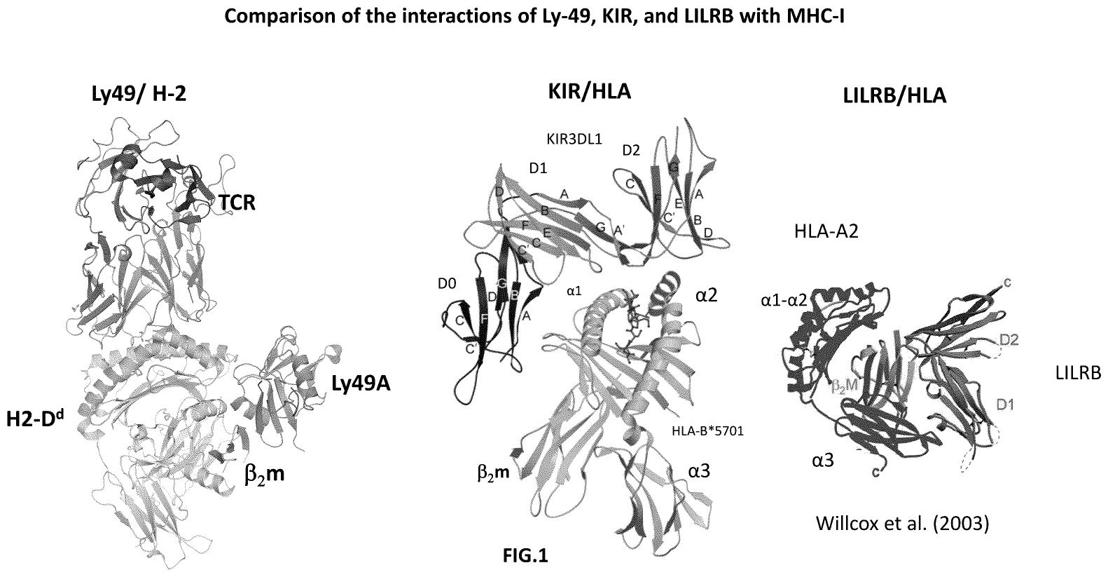

Mouse Ly-49 antigens (members of the C-type-lectin family) are biochemically distinct from human Killer Cell Inhibitor Receptors (KIR, members of the Ig gene superfamily). Second, published crystal structures showed that the binding site of human KIR on HLA antigens is unrelated to the b₂m binding site and overlaps with the TCR binding site (FIG. 1, middle panel). Thus, inhibition of KIR/HLA interactions by anti-HLA would result in inhibition of T cell activation and suppression of the adaptive immune response.

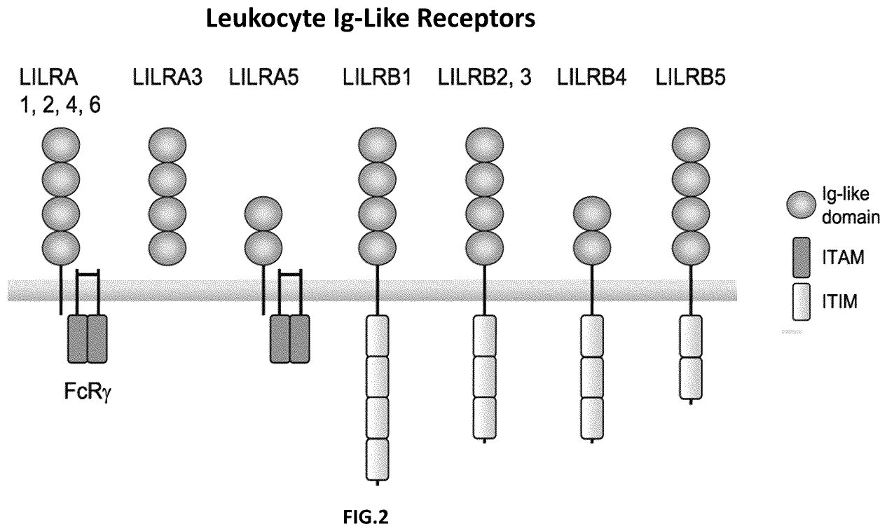

Further review of the literature identified a second group of receptors composed of both activating and inhibitory receptors, termed Leukocyte-Ig-like Receptors (LILRs), some members of which had been shown to interact with a site that includes both HLA heavy chain and b₂m amino acid residues (FIG. 1, right panel). In addition, LILRs react with less variant HLA antigens, including HLA-C, -E, -F, and -G. Members of the LILR family have a much broader tissue distribution than KIR and bind to multiple cell types of the innate and adaptive immune systems as well as certain tumors and non-immune cells (FIG. 2). It was hypothesized that an anti-HLA mAb having binding properties similar to that of the anti-mouse mAb, M1/42, would block the binding of the inhibitory LILRs (LILRBs) to HLA/b₂m complexes and result in activation of the human innate and potentially adaptive immune systems.

### Example 2. Characterization of Anti-HLA-mAbs-W6/32 and Dx17

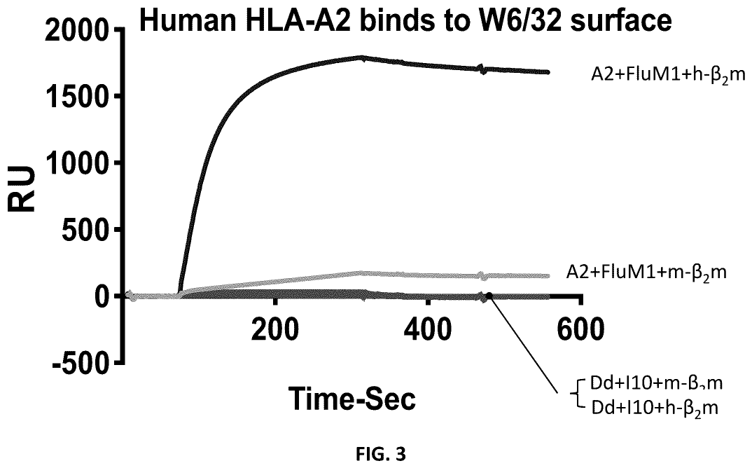

Two pan-HLA mAbs were selected for detailed characterization. W6/32 monoclonal antibody (Cell 14: 9-20, 1978) has been shown to react with all HLA antigens. Furthermore, the binding of W6/32 to HLA was shown to be close to the interaction site of b₂m (J. Immunol. 123, 342, 1979). A second pan anti-HLA, DX17 (Science 268: 403, 1995) was also selected for study. Both W6/32 and DX17 only bound HLA heavy chains in association with human and not mouse b₂m (FIG. 3).

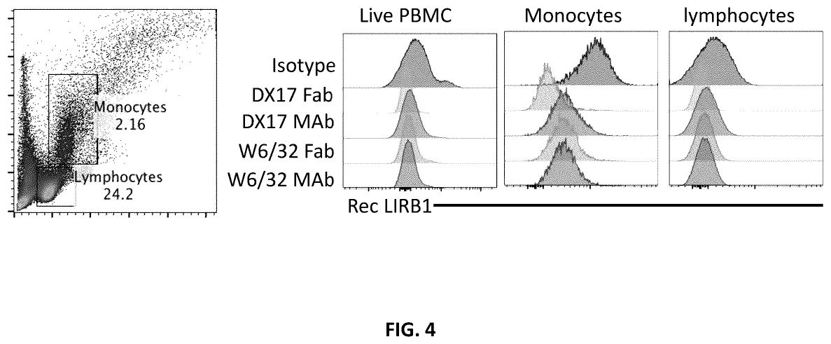

To test the ability of W6/32 monoclonal antibody and DX17 monoclonal antibody, a tetramer of one of the members of the LILRB family (LILRB1) was generated. W6/32 monoclonal antibody and DX17 monoclonal antibody were tested for their ability to inhibit binding of the tetramer to HLA. Both W6/32 and DX17 inhibited the binding of LILRB1 to HLA (FIG. 4).

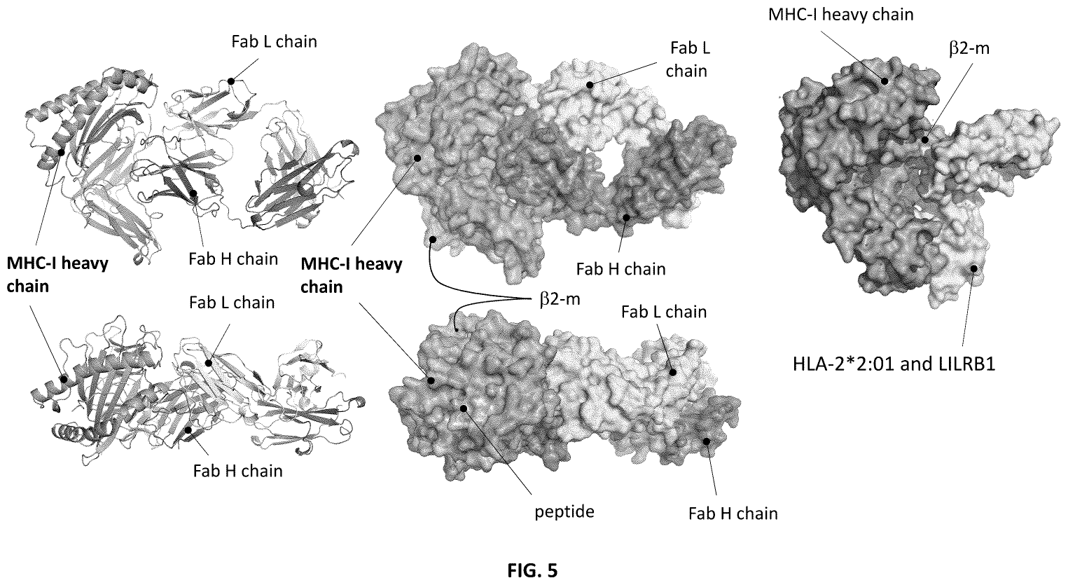

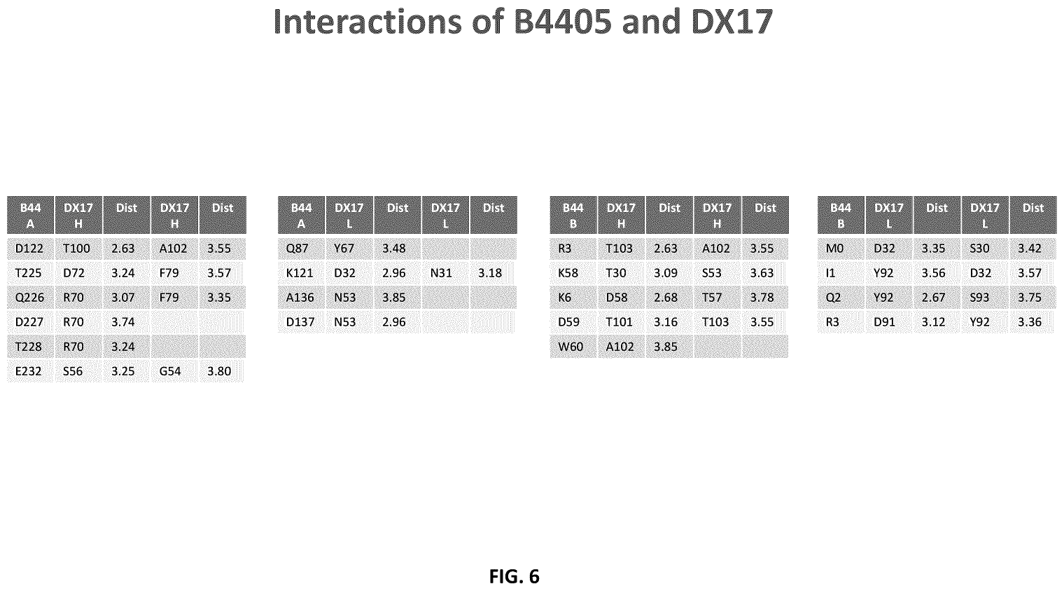

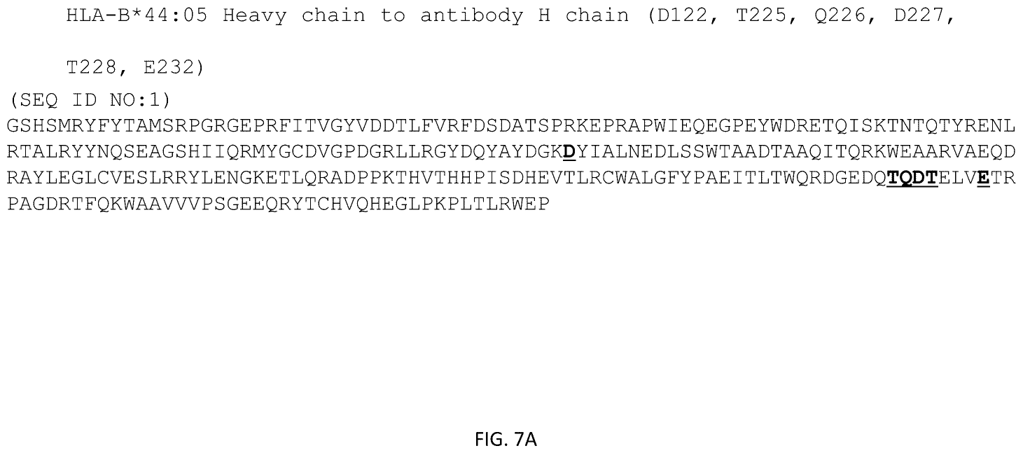

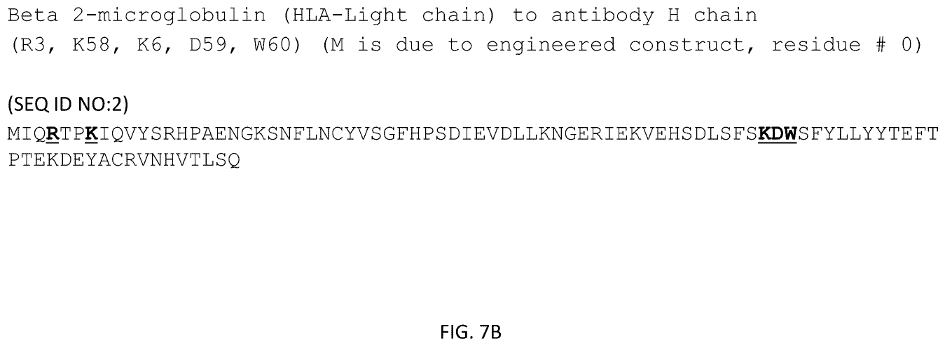

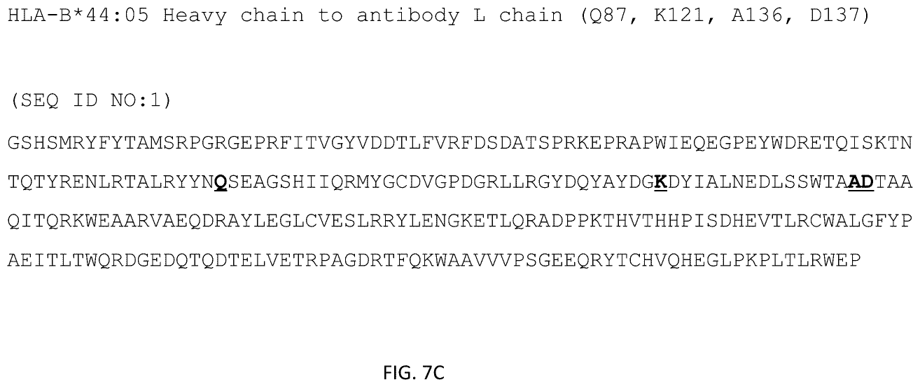

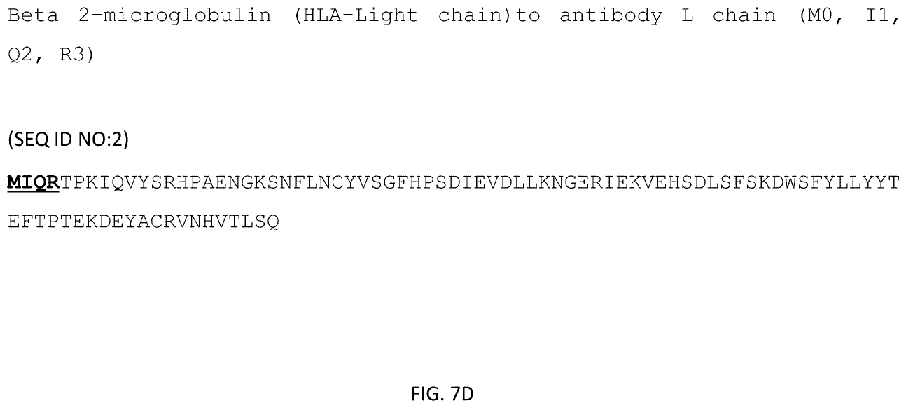

A complex containing a Fab fragment of DX17 monoclonal antibody and one member of the HLA-B family (HLA-B*44:05) were crystalized, and the structure analyzed. The crystal structure of the complex closely resembled the overall structure of LILRB1 and HLA-A*02:01 (FIG. 5). Direct binding of both the heavy and light chains of DX17 to specific amino acids of both the HLA-B heavy chain and the b2m light was demonstrated (FIG. 6).

### Example 3. In Vitro Studies

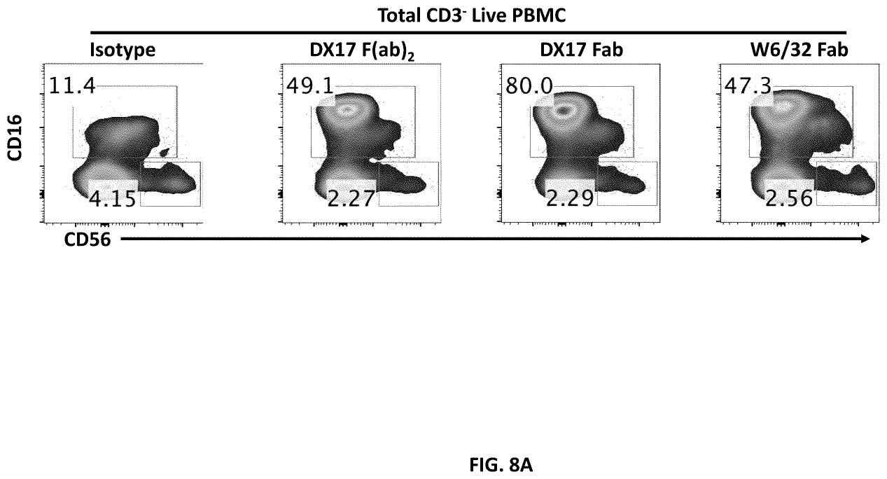

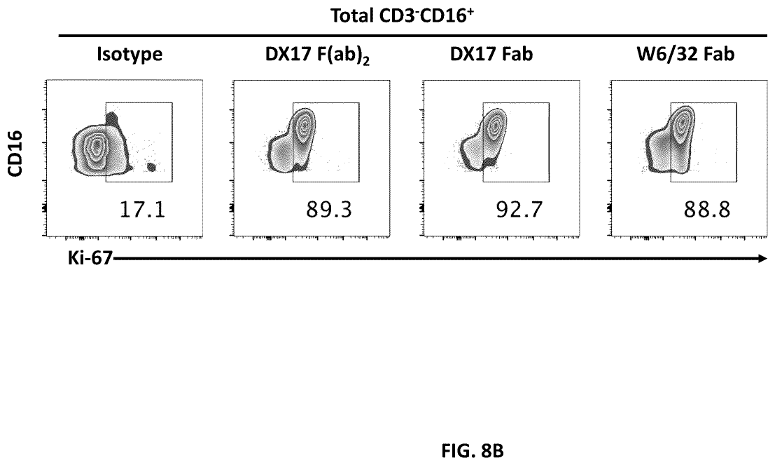

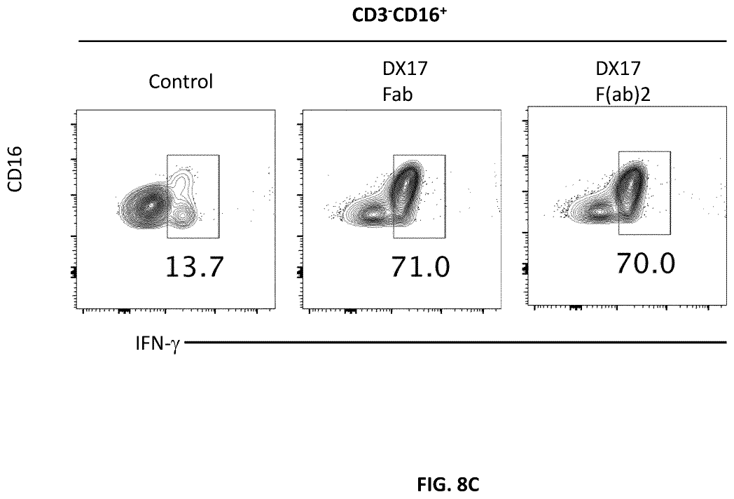

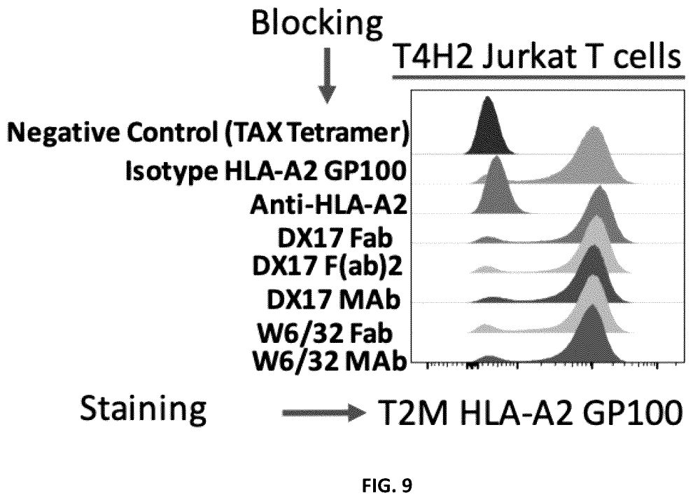

While both W6/32 and DX17 appeared to be excellent candidates for mAbs capable of blocking LILRBs to HLA, it was necessary to develop an in vitro assay to demonstrate that inhibition of LILRB binding resulted in activation of the innate immune system. Human PBMCs were cultured in vitro for 4 days in the presence of either W6/32 or DX17 and assayed for NK cell activation and proliferation. While the intact mAbs did not induce any functional effects, Fab fragments of both mAbs induced NK cell proliferation (as measured by enhanced incorporation of Ki-67) and activation as measured by enhanced production of interferon-g and granzyme B, while a conventional alloantibody to HLA-A2 had no effect (FIGS. 8A-8C). The finding that the Fab fragments of both W6/32 and DX17 were active in the in vitro NK activation assay, suggested that the effects of the mAbs are secondary to blocking rather than signaling. The mAbs did not inhibit the binding of a tumor antigen-specific tetramer to Jurkat cells transfected with an anti-tumor antigen-specific TCR, while binding of the tetramer was inhibited by a conventional anti-HLA-A2 mAb (FIG. 9) Thus, neither W6/32 or DX17 interfere with recognition of antigen by the TCR.

### Example 4. In Vivo Studies

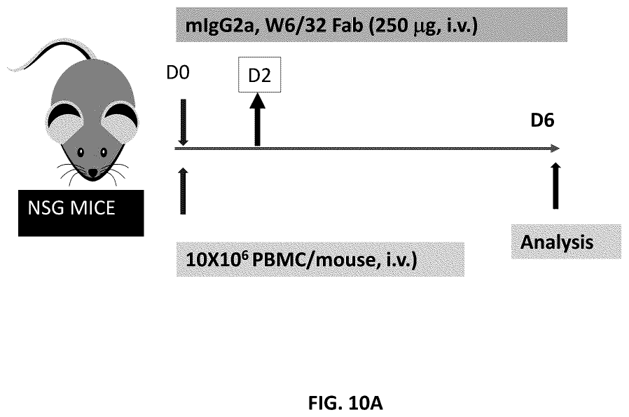

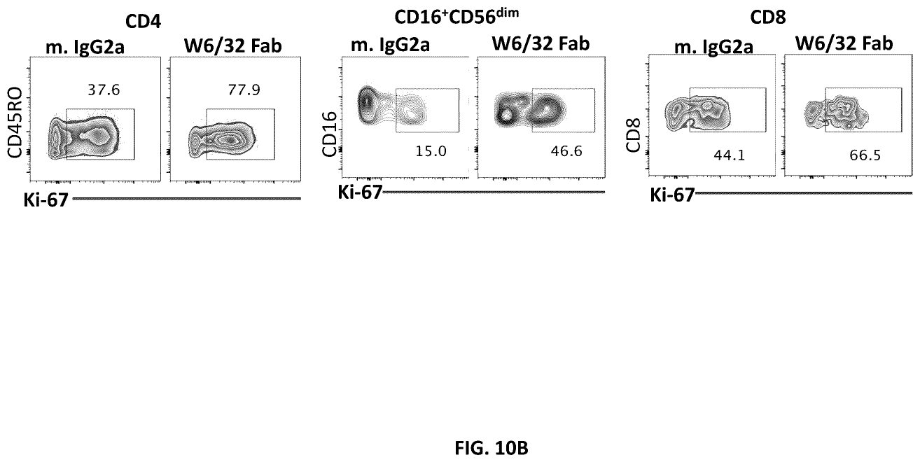

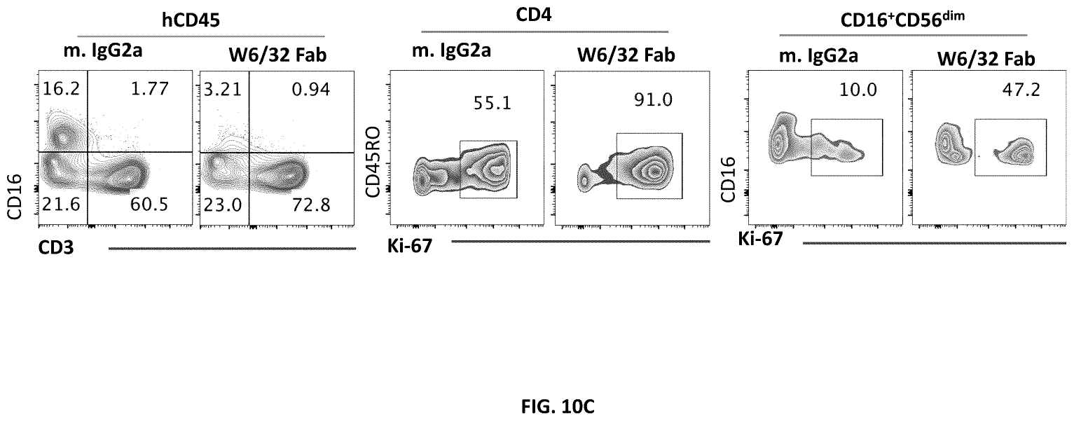

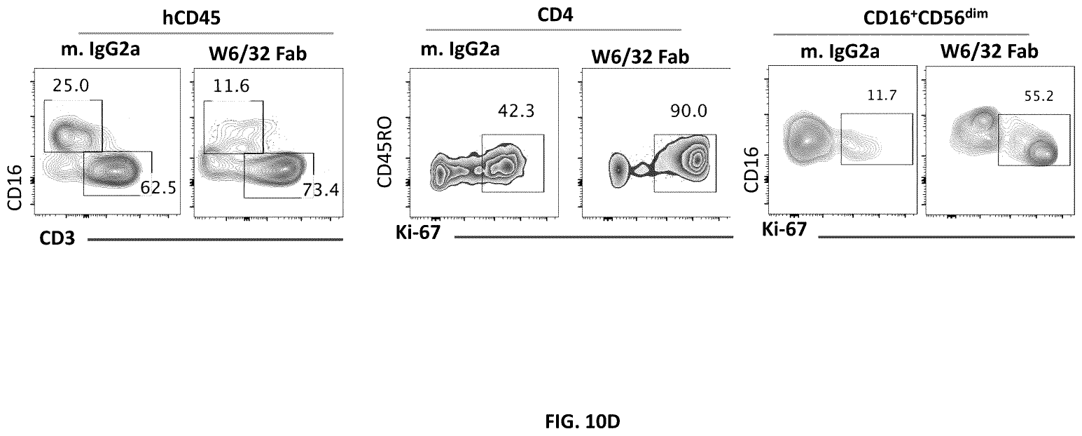

A humanized mouse model was developed to analyze the functional effects of W6/32 and DX17 in vivo. In these studies, immuno-incompetent NSG mice, which lack mouse T, B, and NK cells, were reconstituted with human PBMC. Two days after engraftment, W6/32 Fab was injected intraperitoneally and four days later spleen, liver and lungs were examined for signs of human lymphocyte activation (FIGS. 10A-10D). Markedly enhanced human NK cell proliferation was observed with cells obtained from all 3 sites (FIG. 10B—Spleen; FIG. 10C—liver; FIG. 10D—lung).

### Example 5. Enhancement of NK Cells

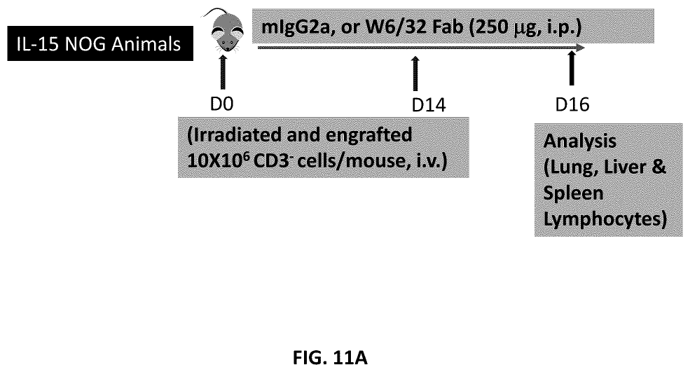

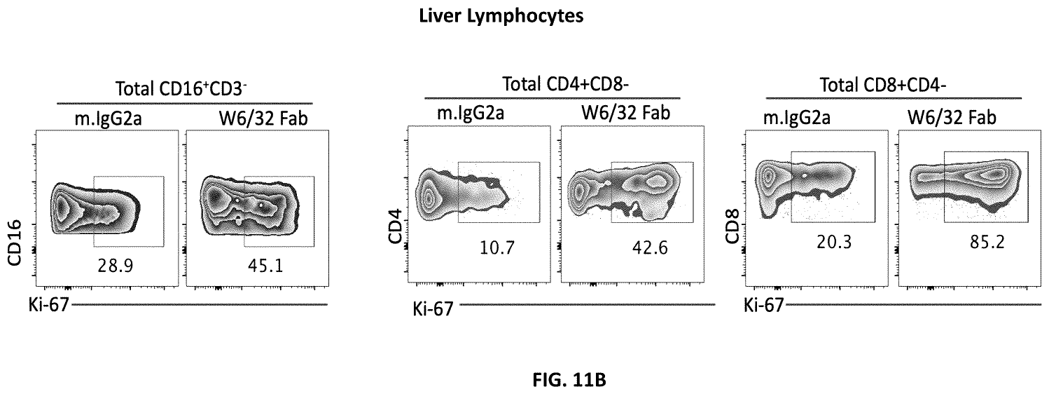

To further study the effects of W6/32 monoclonal antibody, an immuno-incompetent NSG mouse expressing an IL-15 transgene, which has been shown to facilitate engraftment of NK cells, was also utilized. An enriched population of human NK cells was transferred to these mice, and significant engraftment was observed at day 14 post transfer (FIG. 11A). The mice were then treated with W6/32 Fab and 48 hours later monitored for NK activation. A marked enhancement of NK cell proliferation in the liver was observed as assayed by Ki-67 staining (FIG. 11B). The engrafted population contained a small percentage of contaminating CD4+ and CD8+ T cells and an enhancement of the proliferation of these CD4+ and CD8+ T cells was also observed. The results suggest that the anti-pan-HLA mAbs may be used to enhance the NK cell response to tumors. Moreover, a fully humanized version of these mAbs with a non-activating human Fc could be used therapeutically to augment anti-tumor immunity either alone or together with conventional check point inhibitors, to clear chronic infections, to act as an adjuvant to enhance responses to weak immunogens, or to convert potentially harmful allergic reactions (Th-2 immunity) to a less pathogenic Th1-type response. This approach has a major advantage over approaches involving mAbs blocking a single LILRB (e.g., anti-LILRB1), in that that blocking the binding site on MHC-I for all LILRBs should produce a greater enhancement of the activation of the innate immune response and hence a greater therapeutic response.

### Example 6. mAb Fc/NK Cell Interactions

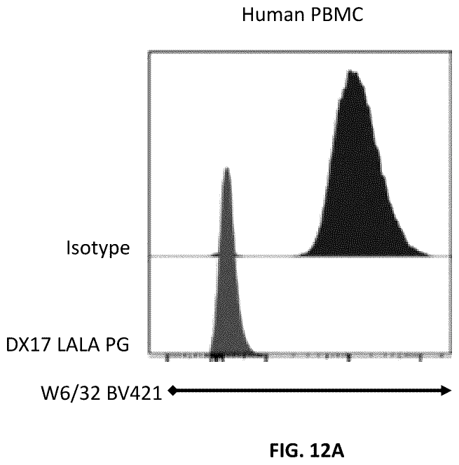

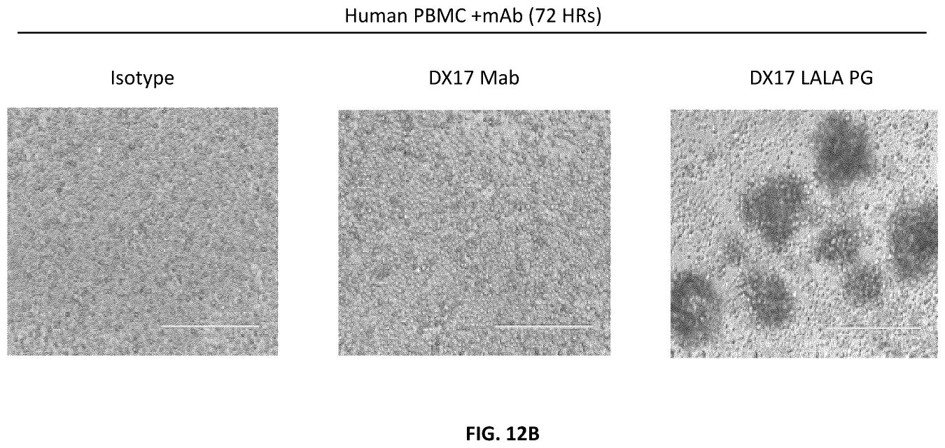

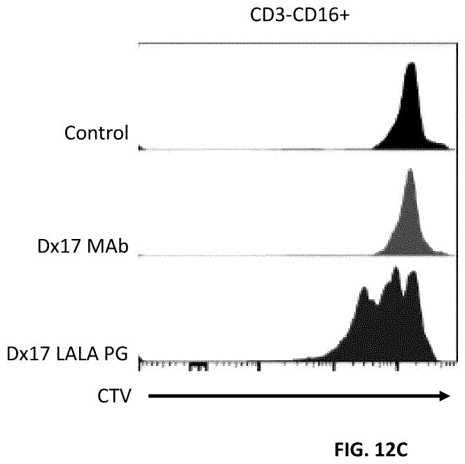

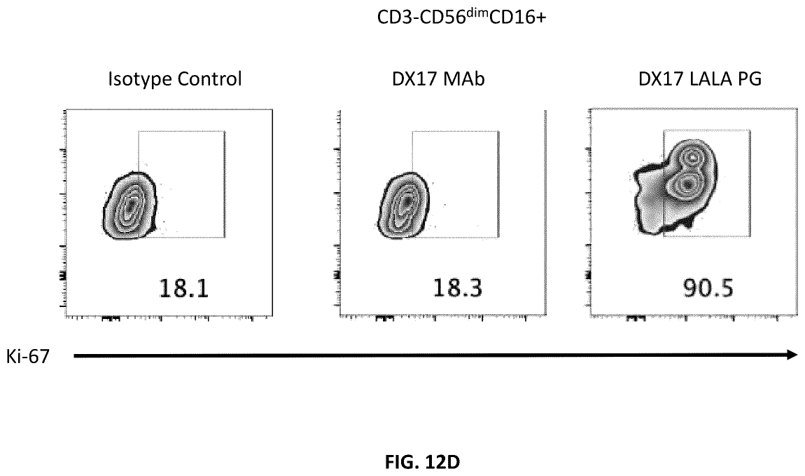

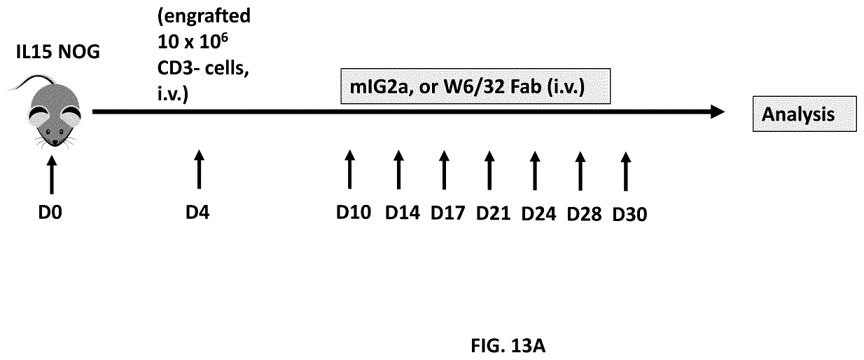

Initial studies demonstrated that the Fab fragment of DX17 (and W6/32) induced proliferation and cytokine production by human NK cells in culture, but no responses were observed when the intact antibodies were used. This observation led to the hypothesis that the Fc fragment of the intact mAbs was interacting with an inhibitory Fc receptor on the responder NK cells, thereby preventing activation. To resolve this issue, a chimeric antibody with a human IgG1 Fc containing three mutations (Leu234ala, leu235ala [LALA], and p329g [PG]) which prevent binding to human Fc receptors and complement protein C1q, was generated. To determine whether the chimeric mAb retained its biologic activity, PBMC were pretreated with the mAb, washed, and then stained with W6/32. As shown in FIG. 12A, the chimeric mAb was active and inhibited the binding of W6/32 to human PBMC. To demonstrate that the chimeric DX17, but not the wild type DX17, retained the same biologic activity as its Fab fragment, the chimeric mAb was added to cultures of human PBMC in the absence of any other stimulus. The chimeric mAb induced activation of the PBMC as indicated by clumping in cell culture (FIG. 12B). Moreover, the chimeric DX17, but not the wild type DX17, induced NK cell proliferation as measured by Ki-67 expression which indicates cell proliferation over the previous 48 hours (FIG. 12C) and dilution of the vital dye cell trace violet (CTV) (FIG. 12D). These studies demonstrate that the biologic activity of the chimeric mAb to induce NK cell activation and proliferation is solely due to its ability to bind to MHC I and not secondary to Fc-FcR signaling. The failure of the intact mAb to induce activation and proliferation of NK cells is secondary to its ability to bind to inhibitory Fc receptors on NK cells and this binding to inhibitory receptors is abolished by the LALA and PG mutations. In addition, the chimeric mAb should have a much longer half-life in vivo than the Fab fragment.

### Example 7. W6/32 Fab Induces Tumor Immunity

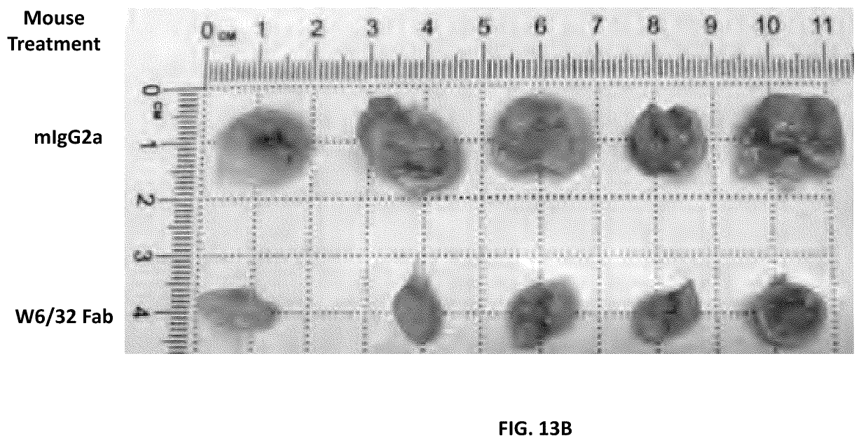

IL-15 transgenic NOG mice were injected subcutaneously with a pancreatic tumor cell line (KLM1) on day 0 and reconstituted with a population enriched for CD3-negative human PBMC (containing B cells, NK cells, monocytes) on day 4. Mice were then treated with W6/32 Fab or control human IgG1 on day 10 and tumor size visualized on D30. FIG. 13A illustrates the experimental design. The results are shown in FIG. 13B. Tumors I the top row are from control treated (IgG2a) mice. Tumors in the bottom row are from mice treated with W6/32 Fab. As seen in FIG. 13B, treatment with W6/32 Fab resulted in a marked reduction in tumor size (bottom row).

1. A method of activating an immune cell that expresses a leukocyte immunoglobulin-like receptor B (LILRB) protein, comprising blocking the interaction of the LILRB protein with an MHC class I HLA molecule by contacting the MHC class I HLA molecule with a compound that recognizes a binding site on the HLA molecule;
wherein binding of the compound to the binding site on the MHC class I HLA molecule inhibits interaction of the MHC class I HLA molecule with the LILRB protein; and,
wherein binding of the compound to the binding site on the MHC class I HLA molecule does not affect the interaction of the MHC class I HLA molecule with a T-cell receptor (TCR).

2.-3. (canceled)

4. The method of claim 1, wherein the compound binds to a location on the HLA molecule that overlaps where the LILRB protein binds the MHC class I HLA molecule.

5. The method of claim 1, wherein the binding site on the HLA molecule comprises one or more amino acid residues selected from the group consisting of:
a glutamine at a position corresponding to amino acid position 87 of SEQ ID NO:1;
a lysine at a position corresponding to amino acid position 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

6. The method of claim 1, wherein the compound comprises a monoclonal antibody (mAb) or an antigen-binding portion thereof.

7. The method of claim 6, wherein the monoclonal antibody, or the antigen-binding portion thereof, inhibits binding of monoclonal antibody W6/32 or monoclonal antibody DX17 to the MHC class I HLA molecule.

8. (canceled)

9. The method of claim 7, wherein the compound is an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDR1 region has the amino acid sequence of SEQ ID NO:4 or SEQ ID NO:12;
wherein the VH CDR2 region has the amino acid sequence of SEQ ID NO:5 or SEQ ID NO:13;
wherein the VH CDR3 region has the amino acid sequence of SEQ ID NO:6 or SEQ ID NO:14;
wherein the VL CDR1 region has the amino acid sequence of SEQ ID NO:8 or SEQ ID NO:16;
wherein the VL CDR2 region has the amino acid sequence of SEQ ID NO:9 or SEQ ID NO:17; and,
wherein the VL CDR3 region has the amino acid sequence of SEQ ID NO:10 or SEQ ID NO:18.

10. (canceled)

11. The method of claim 1, wherein the compound comprises an antibody selected from monoclonal antibody W6/32 and monoclonal antibody DX17.

12. A method of augmenting innate and adaptive immunity in a subject, comprising administering to the subject a compound that recognizes a binding site on the HLA molecule;
wherein binding of the compound to the binding site on the MHC class I HLA molecule inhibits interaction of the MHC class I HLA molecule with the LILRB protein; and,
wherein binding of the compound to the binding site on the MHC class I HLA molecule does not affect the interaction of the MHC class I HLA molecule with a T-cell receptor (TCR).

13. The method of claim 12, wherein the compound binds to a location on the HLA molecule that overlaps where the LILRB protein binds the MHC class I HLA molecule.

14. The method of claim 12, wherein the binding site on the HLA molecule comprises one or more amino acid residues selected from the group consisting of:
a glutamine at a position corresponding to amino acid position 87 of SEQ ID NO:1;
a lysine at a position corresponding to amino acid position 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

15. The method of claim 12, wherein the compound comprises a monoclonal antibody (mAb) or an antigen-binding portion thereof.

16. The method of claim 15, wherein the monoclonal antibody, or the antigen-binding protein thereof, inhibits binding of monoclonal antibody W6/32 or monoclonal antibody DX17 to the MHC class I HLA molecule.

17. The method of claim 15, wherein the compound is an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDR1 region has the amino acid sequence of SEQ ID NO:4 or SEQ ID NO:12;
wherein the VH CDR2 region has the amino acid sequence of SEQ ID NO:5 or SEQ ID NO:13;
wherein the VH CDR3 region has the amino acid sequence of SEQ ID NO:6 or SEQ ID NO:14;
wherein the VL CDR1 region has the amino acid sequence of SEQ ID NO:8 or SEQ ID NO:16;
wherein the VL CDR2 region has the amino acid sequence of SEQ ID NO:9 or SEQ ID NO:17; and,
wherein the VL CDR3 region has the amino acid sequence of SEQ ID NO:10 or SEQ ID NO:18.

18. The method of claim 12, wherein the compound comprises an antibody selected from monoclonal antibody W6/32 and monoclonal antibody DX17.

19. A method of treating a disease in a subject, comprising administering to the subject a compound that recognizes a binding site on the HLA molecule;
wherein binding of the compound to the binding site on the MHC class I HLA molecule inhibits interaction of the MHC class I HLA molecule with the LILRB protein; and,
wherein binding of the compound to the binding site on the MHC class I HLA molecule does not affect the interaction of the MHC class I HLA molecule with a T-cell receptor (TCR).

20. The method of claim 19, wherein the compound binds to a location on the HLA molecule that overlaps where the LILRB protein binds the MHC class I HLA molecule.

21. The method of claim 19, wherein the binding site on the HLA molecule comprises one or more amino acid residues selected from the group consisting of:
a glutamine at a position corresponding to amino acid position 87 of SEQ ID NO:1;
a lysine at a position corresponding to amino acid position 121 of SEQ ID NO:1;
an aspartic acid at a position corresponding to amino acid position 122 of SEQ ID NO:1;
an alanine at a position corresponding to amino acid position 136 of SEQ ID NO:1;
an aspartic acid at a position corresponding to amino acid position 137 of SEQ ID NO:1;
a threonine at a position corresponding to amino acid position 225 of SEQ ID NO:1;
a glutamine at a position corresponding to amino acid position 226 of SEQ ID NO:1;
an aspartic acid at a position corresponding to amino acid position 227 of SEQ ID NO:1;
a threonine at a position corresponding to amino acid position 228 of SEQ ID NO:1;
a glutamic acid at a position corresponding to amino acid position 232 of SEQ ID NO:1;
a methionine at a position corresponding to amino acid position 0 of SEQ ID NO:2;
an isoleucine at a position corresponding to amino acid position 1 of SEQ ID NO:2;
a glutamine at a position corresponding to amino acid position 2 of SEQ ID NO:2;
an arginine at a position corresponding to amino acid position 3 of SEQ ID NO:2;
a lysine at a position corresponding to amino acid position 6 of SEQ ID NO:2;
a lysine at a position corresponding to amino acid position 58 of SEQ ID NO:2;
an aspartic acid at a position corresponding to amino acid position 59 of SEQ ID NO:2; and,
a tryptophan at a position corresponding to amino acid position 60 of SEQ ID NO:2.

22. The method of claim 19, wherein the monoclonal antibody, or the antigen-binding protein thereof, inhibits binding of monoclonal antibody W6/32 or monoclonal antibody DX17 to the MHC class I HLA molecule.

23. The method of claim 19, wherein the compound is an antibody, or an antigen-binding portion thereof, that comprises:
a heavy chain variable region (VH) comprising VH complementarity determining regions (CDRs) 1, 2, and 3; and,
a light chain variable region (VL) comprising VL complementarity determining regions (CDRs) 1, 2, and 3;
wherein the VH CDR1 region has the amino acid sequence of SEQ ID NO:4 or SEQ ID NO:12;
wherein the VH CDR2 region has the amino acid sequence of SEQ ID NO:5 or SEQ ID NO:13;
wherein the VH CDR3 region has the amino acid sequence of SEQ ID NO:6 or SEQ ID NO:14;
wherein the VL CDR1 region has the amino acid sequence of SEQ ID NO:8 or SEQ ID NO:16;
wherein the VL CDR2 region has the amino acid sequence of SEQ ID NO:9 or SEQ ID NO:17; and,
wherein the VL CDR3 region has the amino acid sequence of SEQ ID NO:10 or SEQ ID NO:18.

24. The method of claim 19, wherein the disease is an infectious disease or cancer.
